(19) United States
(12) Patent Application Publication
(10) Pub. No.: US 2013/0231034 A1
(43) Pub. Date: Sep. 5, 2013

(54) METHOD AND APPARATUS FOR PROCESSING LIVESTOCK CARCASSES TO DESTROY MICROORGANISMS

(71) Applicant: TENNANT COMPANY, Minneapolis, MN (US)

(72) Inventors: Charles W. O'Neil, Edina, MN (US); Peter A. Swenson, Minneapolis, MN (US); Bruce F. Field, Golden Valley, MN (US); Larry D. Orstad, Minnetonka, MN (US); Karen R. Wasiluk, St. Paul, MN (US)

(73) Assignee: TENNANT COMPANY, Minneapolis, MN (US)

(21) Appl. No.: 13/774,384

(22) Filed: Feb. 22, 2013

Related U.S. Application Data
(60) Provisional application No. 61/602,970, filed on Feb. 24, 2012.

(51) Int. Cl. A22C 17/08

(52) U.S. Cl. CPC A22C 17/08
USPC 452/173

(57) ABSTRACT

An apparatus and method are provided to clean and sanitize livestock carcasses. The apparatus includes a livestock carcass travel path, at least one liquid dispenser configured to dispense liquid to the carcass travel path, and at least one treatment electrode. A control circuit is configured to cause an alternating electric field to be generated between the electrode and a surface of a carcass along the travel path, through the dispensed liquid.

### CROSS-REFERENCE TO RELATED APPLICATIONS

The present application is based on and claims the benefit of U.S. Provisional Patent Application No. 61/602,970, filed Feb. 24, 2012, the content of which is hereby incorporated by reference in its entirety.

### STATEMENT REGARDING FEDERALLY SPONSORED RESEARCH OR DEVELOPMENT

None.

THE NAMES OF PARTIES TO A JOINT RESEARCH AGREEMENT

None.

### FIELD OF THE DISCLOSURE

The present disclosure relates to processing livestock carcasses such as poultry and other products to deactivate or destroy microorganisms on the carcasses by a mechanism such as electroporation and/or electrohydraulic shock. In one particular example, the disclosure relates to applying an electrical potential to microorganisms on the surface of a carcass through a liquid delivered to the carcass by an apparatus.

### BACKGROUND OF THE DISCLOSURE

A typical poultry processing plant includes an overhead conveyor in which live birds are hung by their feet from a shackle and carried by the conveyor through a series of processing stations. For example, the birds are stunned, bled, scalded, defeathered, eviscerated, rinsed and chilled.

Carcasses of poultry are typically scalded after slaughter to facilitate removal of feathers. At the scalding station, the bird carcasses are immersed in hot water or steam. After scalding, the carcasses are passed through the picker, which includes a large number of rubber fingers that beat against the carcass to remove the feathers. The carcass is then eviscerated, cleaned and chilled.

The various processing steps contaminate the carcasses by release bacteria from the feathers, internal organs and intestines of the carcasses as they pass through the stations.

In an effort to reduce the bacterial loads on the carcasses, the carcasses are often passed through one or more rinsing stations between various steps of the process. It has been found that rinsing the carcasses is only partially effective in disinfecting the carcasses. Therefore, chemicals are often used to improve the disinfecting process. For example, the carcasses may be immersed in a bath or sprayed with a liquid containing chlorine or another chemical to improve disinfection. However, this leaves the carcasses with chlorine or other chemical residue, which is considered by the present inventors as being undesirable. Similar problems occur when processing other types of livestock carcasses.

### SUMMARY

An illustrative aspect of the present disclosure is directed to an apparatus, which includes a livestock carcass travel path, at least one liquid dispenser configured to dispense liquid to the carcass travel path, and at least one treatment electrode. A control circuit is configured to cause an alternating electric field to be generated between the electrode and a surface of a carcass along the travel path, through the dispensed liquid.

Another illustrative aspect of the present disclosure is directed to a method, which includes receiving a livestock carcass along a travel path. A liquid is dispensed from at least one liquid dispenser to the carcass along the travel path, so as to create an electrically conductive path from the liquid dispenser to the carcass. During the step of dispensing, an alternating electric field is generated through the liquid along the conductive path, wherein the electric field is applied to the liquid with a treatment electrode and is sufficient to destroy at least one microorganism on a surface of the carcass.

Another illustrative aspect of the present disclosure is directed to a poultry rinse cabinet. The cabinet includes a poultry carcass travel path extending through the rinse cabinet, and at least one liquid flow path. First and second sets of spray nozzles are positioned on first and second opposing sides of the carcass travel path, each spray nozzle in the first and second sets being coupled to at least one of the liquid flow paths and being oriented to direct a respective spray output toward the carcass travel path. The cabinet also includes a respective treatment electrode for each of the spray nozzles in the first and second sets, wherein each treatment electrode is electrically coupled to at least one of the respective liquid travel path or the respective spray output. A control circuit is configured to cause an alternating electric field to be generated between each of the treatment electrodes and the carcass travel path, through the respective spray outputs, which is sufficient to destroy at least one microorganism on a surface of a carcass along the travel path.

### BRIEF DESCRIPTION OF THE DRAWINGS

FIG. 1 is a diagram illustrating a livestock carcass processing system, with a plucker and a rinse cabinet positioned along a conveyor.

FIG. 1A is a top plan, diagrammatic view of the processing system shown in FIG. 1.

FIG. 2 is a simplified, schematic diagram of the rinse cabinet.

FIG. 3 is a waveform diagram illustrating an example of the voltage pattern applied to an electrolysis cell in the rinse cabinet according to an exemplary aspect of the present disclosure.

FIG. 4 is an exploded view of a nozzle within the rinse cabinet, which has an attached high-voltage electroporation electrode according to an illustrative embodiment of the disclosure.

FIG. 5A is a diagram illustrating an example of conductive paths formed between a spray nozzle and a surface by an electrically charged output spray.

FIG. 5B is a diagram illustrating an example of an electroporation mechanism, whereby a cell suspended in a medium is subjected to an electric field.

FIG. 5C is a diagram illustrating an example of a cell membrane having pores expanded by electroporation.

FIG. 6 is an example of a waveform diagram illustrating the voltage pattern applied to an electroporation electrode in the rinse cabinet according to an exemplary aspect of the present disclosure.

FIG. 7 is a block diagram of an example of a control circuit for controlling electrolysis cell(s) in the rinse cabinet according to an exemplary aspect of the disclosure.

FIG. 8 is a block diagram of an example of a control circuit for controlling the electroporation electrode(s) in the rinse cabinet according to an exemplary aspect of the disclosure.

FIG. 9A is a perspective view of an electrolysis cell according to an exemplary aspect of the disclosure, which can be used in the rinse cabinet shown in FIG. 1.

FIG. 9B is a cross-sectional view of the electrolysis cell taken along lines 9B-9B of FIG. 9A.

FIG. 10A is a perspective view of a prototype rinse cabinet according to an exemplary aspect of the present disclosure.

FIG. 10B is a side elevation view of the prototype rinse cabinet shown in FIG. 10A.

FIG. 10C is a partial elevation view of a first side of the prototype rinse cabinet shown in FIG. 10A.

FIG. 10D is a partial elevation view of a second, opposing side of the prototype rinse cabinet shown in FIG. 10A.

FIG. 10E is an end elevation view of the prototype rinse cabinet shown in FIG. 10A.

### DETAILED DESCRIPTION OF ILLUSTRATIVE EMBODIMENTS

The following is provided as a description of examples of one or more aspects of the present disclosure. The below detailed description and above-referenced figures should not to be read as limiting or narrowing the scope of the invention as will be claimed in issued claims. It will be appreciated that other embodiments of the invention covered by one or more of the claims may have structure and function which are different in one or more aspects from the figures and examples discussed herein, and may embody different structures, methods and/or combinations thereof of making or using the invention as claimed in the claims, for example.

Also, the following description is divided into sections with one or more section headings. These sections and headings are provided for ease of reading only and, for example, do not limit one or more aspects of the disclosure discussed in a particular section and/or section heading with respect to a particular example and/or embodiment from being combined with, applied to, and/or utilized in another particular example, and/or embodiment which is described in another section and/or section heading. Elements, features and other aspects of one or more examples may be combined and/or interchangeable with elements, features and other aspects of one or more other examples described herein.

An aspect of the present disclosure for example relates to sanitizing livestock carcasses by applying an output fluid (including a liquid stream and/or a gas/liquid mixture, water vapor, gaseous liquid, mist, spray or aerosol mixture for example), which has enhanced sanitizing properties, to the carcass. The sanitizing properties are enhanced, for example, by applying an electric field, such as an alternating electric field, to the surface of the carcass through the output fluid. The electric field applied to the carcass, and thus to cells of microorganisms on a carcass, meets or surpasses a threshold such that the cells become permanently damaged by a process known as irreversible electroporation, for example. If the electric field threshold is reached or surpassed, electroporation will compromise viability of the cells, resulting in irreversible electroporation. Thus, one or more examples of the present disclosure deliver an applied electric field to the carcass through a charged output liquid.

In one or more examples, the microorganisms are suspended from the surface of the carcass by liquid dispensed from spray nozzles and through which the electric field is applied. Such suspension can be enhanced, for example by electrochemically activating the output liquid with an electrolysis cell, for example, which alters the oxidation-reduction potential of the liquid to exceed about +/−50 millivolts, for example. The electrolysis cell can, for example, increase the ORP of a liquid to aid in suspension of the microorganisms through the action of charged nanobubbles, for example. Other mechanisms can also be used to alter a liquid's ORP and/or enhance suspension of particles and microorganisms from a surface.

In a particular example, the output fluid is applied to the carcasses at one or more stages in a livestock processing plant as the livestock carcasses are moved along one or more conveyors through the various stages of the plant.

1. Example Processing System

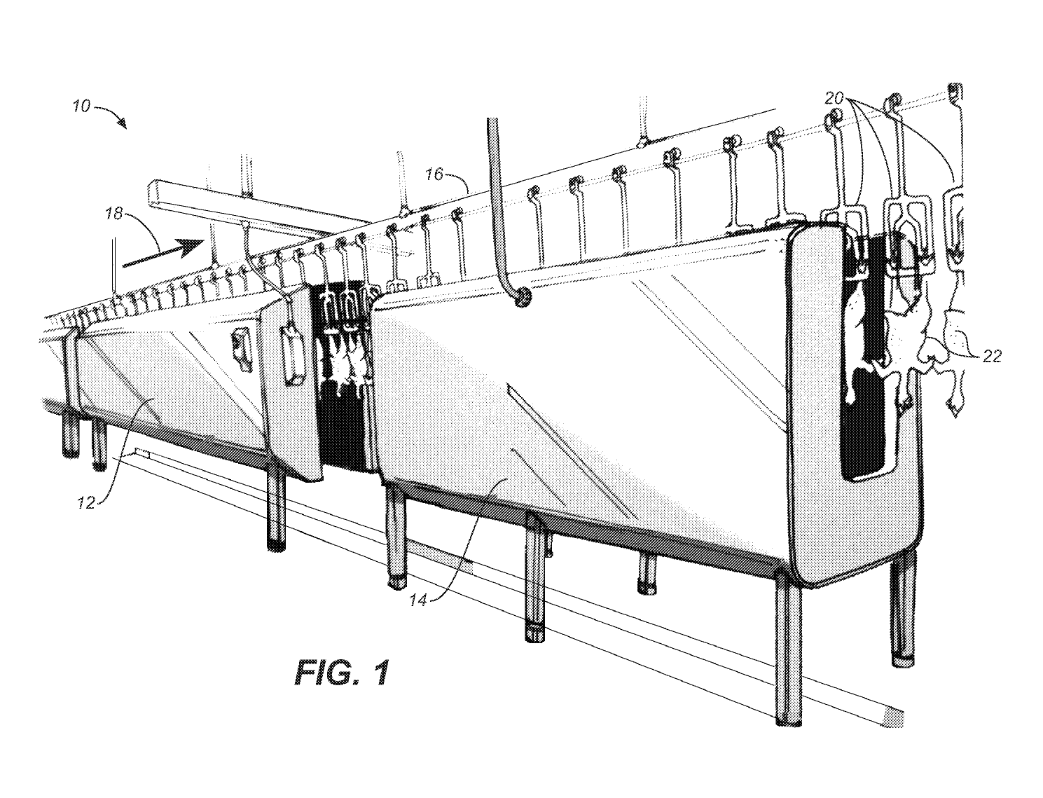

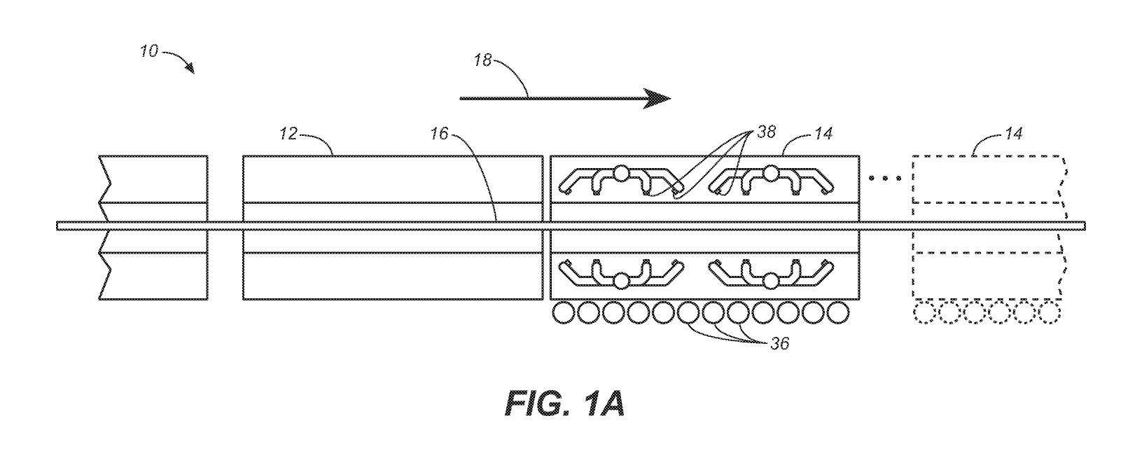

FIG. 1 is a diagram illustrating a livestock carcass processing system 10 according to an illustrative aspect of the present disclosure in which the livestock carcasses are treated by an electric field applied through an output spray as the carcasses pass through the system. In addition, for example, the output spray has enhanced suspension properties for suspending bacteria and other microorganisms from the surface of the carcasses as the microorganisms are treated by the electric field.

In the example shown in FIG. 1, the system 10 illustrates a portion of a poultry processing plant, which includes a picker 12 and a rinse cabinet 14. An overhead conveyor 16 moves poultry carcasses through the various stages of the plant in the direction of arrow 18. The overhead conveyor 16 carries a plurality of shackles 20 on which the poultry carcasses 22 are hung. In one particular example, the shackles 20 are spaced about 6 inches apart from one another along conveyor 16, as measured center-to-center. Other distances can also be used. The conveyor can be configured to move the carcasses through picker 12 and rinse cabinet 14 at any suitable rate, such as 50-300 feet per minute, and at 110 feet per minute processes about 13,200 carcasses per hour.

Picker 12 can include any suitable picker, such as a picker having a large number of rubber fingers (also called whips) that beat against the carcasses to remove the feathers as the carcasses pass through the picker. For example, the rubber fingers may be mounted on one or more rotating shafts, drums or heads, which are positioned within the cabinet so the fingers strike the carcasses as they pass. For example, the shafts, drums or heads can be positioned on opposite sides of the cabinet and can be counter-rotating.

Rinse cabinet 14 can have any length, such as 12-18 feet long, and includes an array of spray nozzles for spraying an output fluid (such as tap water or electrolyzed tap water) onto the carcasses as the carcasses pass through the cabinet. As explained in more detail below, the electrolyzed water applied to the carcasses assists in suspending microorganisms and dirt from the surface of the carcasses. Electrolysis can, for example, increase the ORP of a liquid to aid in suspension of the microorganisms through the action of charged nanobubbles, for example. Other mechanisms, such as an additive, can be used to alter a liquid's ORP and/or enhance suspension of particles and microorganisms from a surface.

In order to enhance the sanitizing properties of the output liquid that is applied to the carcasses, an electric field is applied to carcasses through the output liquid. For example, the electric field may be applied through one or more high voltage “electroporation electrodes”, which makes electrical contact with the output liquid at any suitable location along the liquid flow path. In a particular example, the electrodes are placed as near as possible to the spray output. If the nozzle is electrically conductive, the electrode may be connected directly to the nozzle, for example. A respective electrode may be attached to each of the nozzles in rinse cabinet 14. In another example, a respective electrode is electrically connected to an electrically-conductive barb that is inserted into the flow path leading to each nozzle. In a further embodiment, an electrode can be positioned within the output spray, itself, following the nozzle. Although the term “electroporation electrode” is used in the description to refer to an electrode that applies an electric field through the output liquid, this term is used for convenience only and is not intended to limit its operation or effect on microorganisms to a process of electroporation.

In an example, the various nozzles in rinse cabinet 14 are positioned such that all or substantially all exterior surfaces of each carcass are directly contacted with an output spray and to maintain consistent contact with the various output sprays as the carcasses are moved along the travel path through rinse cabinet 14. The length of cabinet 14, the number and location of spray nozzles, their orientations and the travel rate of the carcasses can be adjusted to achieve a desired “contact time” with the output sprays, and thus the applied electric fields, within the rinse cabinet.

In one aspect of the present disclosure, rinse cabinet 14 is positioned relative to picker 12 such that the poultry carcasses are treated by in rinse cabinet as soon as possible after the carcasses are plucked by picker 12. It has been found that bacteria deposited on the skin of a plucked carcass quickly begins to attached and absorb into the skin, thus making it more difficult to sanitize the carcass through a rinse process, even if the rinse process applies a chemical agent, such as chlorine.

In one example, the physical spacing of rinse cabinet 14 from picker 10 and the rate of conveyor 16 are set such that each carcass 22 is treated with an applied electric field, through an output spray, within a predetermined time period of exiting picker 12, such as zero to 30 seconds, or zero to 10 seconds. Other time periods can also be used. This reduces the efficacy requirements of the cleaning and/or sanitizing process performed by rinse cabinet 14. For example, it may be desirable for a particular processing plant to achieve at least a 1 log₁₀ bacterial reduction relative to untreated water only in rinse cabinet 14 if the carcasses are treated within a predetermined time of plucking. Based on the rate of advancement and the length of rinse cabinet 14, each carcass may have a treatment time within cabinet 14 of approximately 6 seconds to 24 seconds, for example.

In another example, rinse cabinet 14 is combined with spray cabinet 12. The spray nozzles may be stationary relative to the rubber fingers, and are positioned to apply the output spray and electric field to the carcasses as the carcasses are beat by the rubber fingers. In another example, the spray nozzles are positioned within picker cabinet 12 before and/or after the rubber fingers, relative to the direction of movement 18 of conveyor 16.

Rinse cabinet 14 may have housing walls, such as shown in FIG. 1, which partially enclose, substantially enclose or completely enclose the carcass travel path. In other embodiments, rinse cabinet 14 has no enclosure.

FIG. 1A is a top plan, diagrammatic view of processing system 10, with plucker 12 and rinse cabinet 14 positioned along conveyor 16. Rinse cabinet 14 includes a plurality of spray nozzles 38 positioned on opposing sides of the carcass travel path through the cabinet. One or more electrolysis cells 36 feed spray nozzles 38 with electrolyzed liquid (e.g., electrolyzed tap water) for spraying onto the carcasses. As illustrated in greater detail below, one or more of the nozzles 38 includes an electrode for applying an electric field to the carcasses through the spray output. For example, each nozzle 38 includes a respective electrode.

The number, arrangement and grouping of nozzles, electrodes and electrolysis cells in rinse cabinet 14 can be selected as desired. The arrangement shown in FIG. 1A is provided as a simplified example only. An exemplary commercial embodiment may include a larger number of nozzles, electrodes and electrolysis cells than shown in FIG. 1A.

2. Simplified Block Diagram of Rinse Cabinet

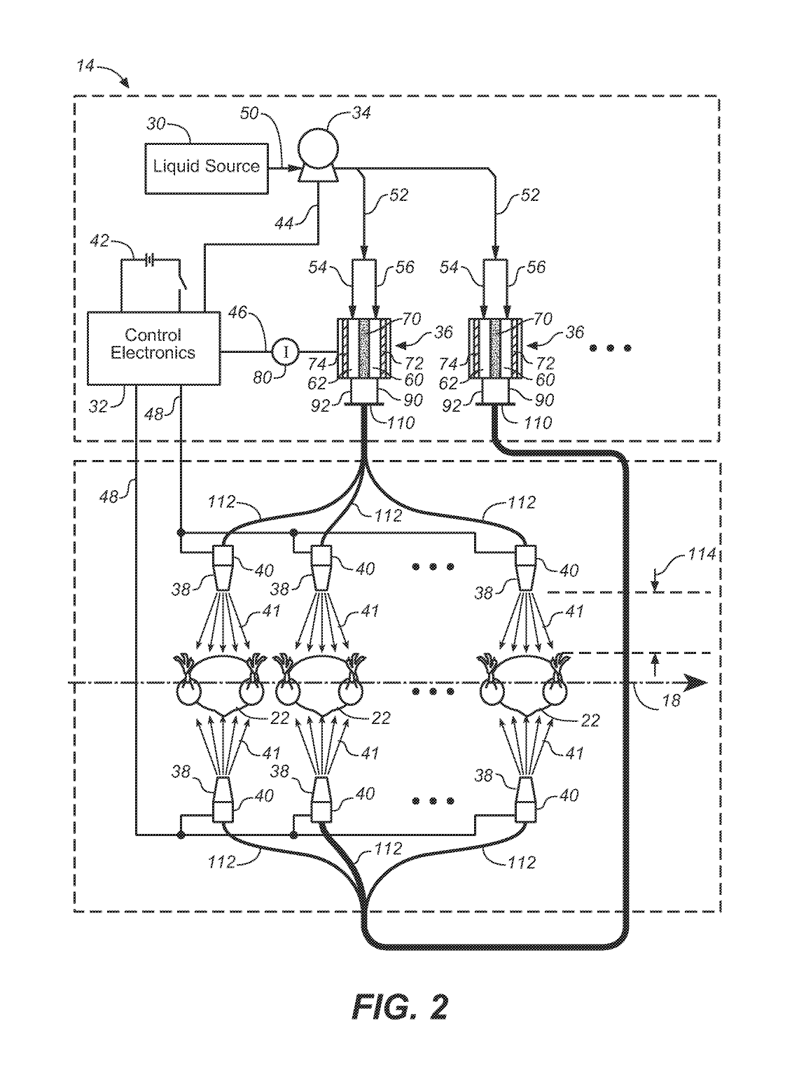

FIG. 2 is a simplified, schematic diagram of rinse cabinet 14. As shown, rinse cabinet 14 includes liquid source 30, control electronics 32, pump 34, one or more electrolysis cells 36, and one or more liquid dispensers (e.g., nozzles) 38 and associated electroporation electrodes 40.

Liquid source 30 may include a reservoir or a fluid line coupling for containing and/or receiving a feed liquid to be treated and then applied to the livestock carcasses or other products through nozzles 40 as the carcasses are conveyed passed the nozzles by conveyor 16 (shown in FIG. 1) along the direction of arrow 18. Rinse cabinet 14 may include nozzles 38 positioned on one side, opposite sides, or multiple sides of carcass 22. In some embodiments, the feed liquid may include one or more additives, such as electrolytic compositions (e.g. salts), which are desirably dissolved or otherwise suspended in the feed liquid. In other embodiments, the feed liquid may consist essentially of tap water. The following discussion of the cleaning systems of the present disclosure (e.g., cleaning system 10) is made with reference to water (e.g., tap water) as the feed liquid with the understanding that the cleaning systems of the present disclosure may be used with a variety of different feed liquids.

Control electronics 32 may include one or more printed circuit boards containing electronic devices for powering and controlling the operation of pump 34, electrolysis cells 36, electroporation electrodes 40, and other suitable components of rinse cabinet 14, for example. For example, control electronics 32 may apply electrical power from electrical source 42 to pump 34, electrolysis cell 36, and electrodes 40, respectively over electrical lines 44, 46, and 48 during operation.

In one embodiment, control electronics 42 simultaneously applies electrical power to pump 34, electrolysis cell 36, and electrodes 40. This embodiment is beneficial for providing an on-demand activation of pump 34, electrolysis cell 36, and electrodes 40, such as when a plant control system for processing system 10 (shown in FIG. 1) and/or plant operator actuates a control mechanism (such as a switch) to activate control electronics 32 during motion of conveyor 16. Alternatively, control electronics 32 may apply electrical power independently to pump 34, electrolysis cell 36, and/or electrodes 40.

Pump 34 may include one or more liquid pumps operated by control electronics 32 to draw the feed water from liquid source 30 through fluid lines 50 and 52 at a predetermined flow rate and/or pressure. The predetermined flow rate and/or pressure may be based on a fixed pumping rate, or may be adjustable by control electronics 32 over electrical line 44, thereby allowing the flow rate and/or pressure within line 50 of the feed water to be adjusted.

In the shown embodiment, pump 34 is located downstream from liquid source 30 and upstream from electrolysis cell 36 for drawing water from liquid source 30 to electrolysis cell 36. In alternative embodiments, pump 34 may be positioned at any suitable location along the flow path between liquid source 30 and nozzles 40.

2.1 Electrolysis

Rinse cabinet 14 includes one or more electrolysis cells 36, which receive the pumped feed water from pump 34 over fluid lines 52, which split into inlet lines 54 and 56 prior to (or after) entering electrolysis cells 36. In particular example, a first portion of the feed water may flow through inlet line 54, and is directed into anode chamber 60 of electrolysis cell 36. Correspondingly, a second portion of the feed water in inlet line 56 is directed into cathode chamber 62 of electrolysis cell 36. Rinse cabinet 14 may include multiple electrolysis cells 36 arranged serially and/or in parallel with one another. In one particular example, rinse cabinet 14 includes six electrolysis cells 36 connected together in parallel with one another.

In one embodiment, each electrolysis cell 36 includes a barrier 70, an anode electrode 72, and a cathode electrode 74, where barrier 70 includes a membrane or other diaphragm that separates anode chamber 60 and cathode chamber 62. Anode electrode 72 includes one or more electrodes located in anode chamber 60. Correspondingly, cathode electrode 74 includes one or more electrodes located in cathode chamber 62.

Barrier 70 has pores in a range of about 1 micron to about 200 microns, for example. With small pores sizes, the barrier can act as a selective ion exchange membrane. In embodiments in which barrier 70 includes an ion exchange membrane, barrier 70 can include a cation exchange membrane (i.e., a proton exchange membrane) or an anion exchange membrane. Suitable cation exchange membranes for barrier 70 include partially and fully fluorinated ionomers, polyaromatic ionomers, and combinations thereof. Examples of suitable commercially available ionomers for barrier 70 include sulfonated tetrafluorethylene copolymers available under the trademark “NAFION” from E.I. du Pont de Nemours and Company, Wilmington, Del.; perfluorinated carboxylic acid ionomers available under the trademark “FLEMION” from Asahi Glass Co., Ltd., Japan; perfluorinated sulfonic acid ionomers available under the trademark “ACIPLEX” Aciplex from Asahi Chemical Industries Co. Ltd., Japan; and combinations thereof.

In another embodiment, barrier 70 includes a material that does not act as a selective ion exchange membrane, but maintains general separation of the anode and cathode compartments. In one particular example, the barrier material includes pores having diameters of about 100-110 microns, whereas typical pore sizes of a selective ion exchange membrane may be about 1 micron in diameter, for example. These large pores conduct current between the anode and cathode electrodes and facilitate production of bubbles in the output liquid. Exemplary materials for such a barrier include polypropylene, polyester, nylon, PEEK mesh, Polytetrafluoroethylene (PTFE), and thermoplastic mesh, for example. In a particular example, the barrier material includes polypropylene having a thickness of 10 mils (0.254 mm). Other materials and material thicknesses can also be used.

In a further embodiment, electrolysis cells 36 contain no barrier between the anode and cathode electrodes. This embodiment is believed to reduce the resistance between the cell electrodes and therefore increase the current applied to the liquid passing through the cell. Increasing the applied current is believed to favorably increase the amount of dissolved hydrogen and oxygen in the treated liquid.

Electrodes 72 and 74 can be made from any suitable material, such as titanium and/or titanium coated with a precious metal, such as platinum, or any other suitable electrode material. The electrodes and respective chambers can have any suitable shape and construction. For example, electrodes 72 and 74 can be flat plates, coaxial plates, rods, or a combination thereof, and may be solid or mesh (i.e., porous). In one specific example, the mesh is formed of 0.023-inch diameter T316 (or, e.g. 304) stainless steel having a grid pattern of 20×20 grid openings per square inch. In other embodiments the electrodes include titanium with an iridium oxide, platinum or white gold coating, for example. In one specific example, the electrodes are titanium mesh electrodes with an iridium oxide coating and are spaced apart from one another by a gap of about 15-50 thousandths of an inch (0.015 inch to 0.050 inch; or 0.38 mm to 1.27 mm), such as 0.030 inches (0.76 mm). Alternatively, one or both electrodes may be solid. Other dimensions, arrangements and materials can be used in other examples.

Electrodes 72 and 74 are electrically connected to opposite terminals of a power supply, such as electrical source 42, through control electronics 32 and electrical line 46. During operation, control electronics 32 may apply a voltage potential across anode electrode 72 and cathode electrode 76. Control electronics 32 can provide a constant DC output voltage, a pulsed or otherwise modulated DC output voltage, and/or a pulsed or otherwise modulated AC output voltage to electrodes 72 and 74, for example. In the shown embodiment, rinse cabinet 14 may also include current sensor 80 located along electrical line 46 and/or within electrolysis cell 36 to detect the intensity of the current drawn through electrolysis cell 36.

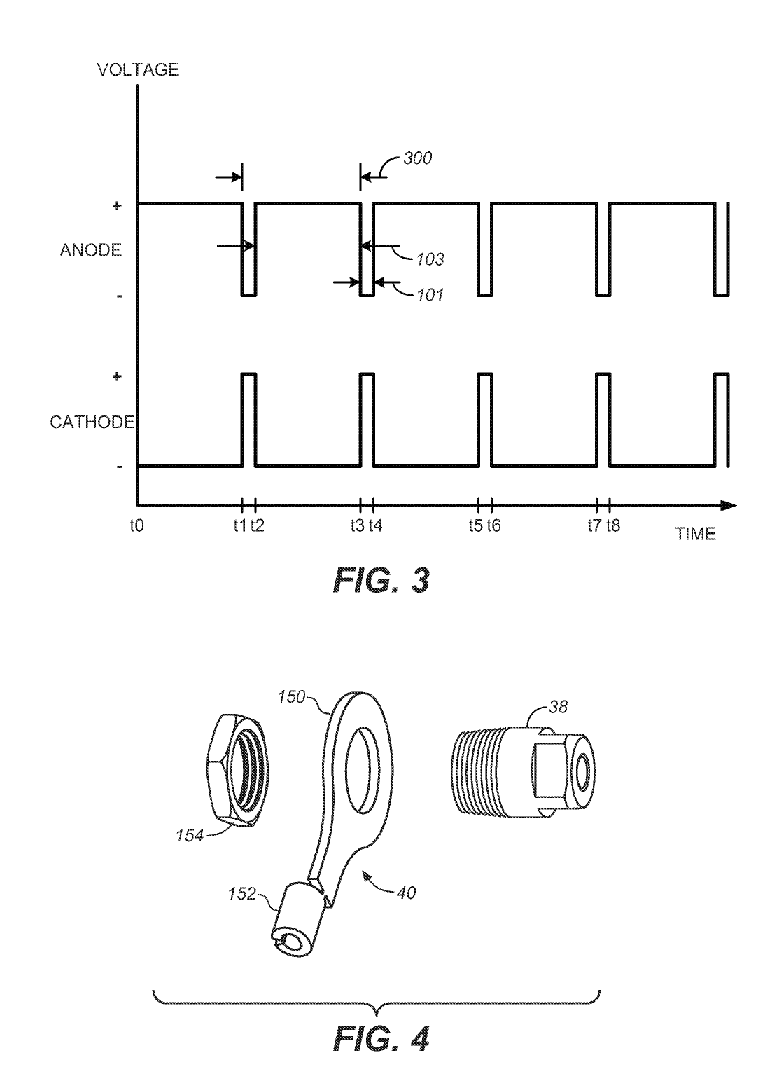

FIG. 3 is a diagram, which illustrates the voltage pattern applied to the anode and cathode of electrolysis cell 36 according to an exemplary aspect of the present disclosure. A substantially constant, relatively positive voltage is applied to the anode, while a substantially constant, relatively negative voltage is applied to the cathode. However, periodically each voltage may be briefly pulsed to a relatively opposite polarity to repel scale deposits. In some examples, there is a desire to limit scale deposits from building on the electrode surfaces. In this example, a relatively positive voltage is applied to the anode and a relatively negative voltage is applied to the cathode from times t0-t1, t2-t3, t4-t5 and t6-t7. During times t1-t2, t3-t4, t5-t6 and t7-t8, the voltage applied to each electrode is reversed. The reversed voltage level can have the same magnitude as the non-reversed voltage level or can have a different magnitude if desired.

The frequency of each brief polarity switch can be selected as desired. As the frequency of reversal increases, the amount of scaling decreases. However, the electrodes may lose small amounts of platinum (in the case of platinum coated electrodes) with each reversal. As the frequency of reversals decreases, scaling may increase. In one example, the time period between reversals, as shown by arrow 100, is in the range of about 1 second to about 600 seconds. Other periods outside this range can also be used. In this example, the time period of normal polarity 103, such as between times t2 and t3, is at least 900 milliseconds.

The time period at which the voltages are reversed can also be selected as desired. In one example, the reversal time period, represented by arrow 102, is in the range of about 50 milliseconds to about 100 milliseconds. Other periods outside this range can also be used.

With these ranges, for example, each anode chamber produces a substantially constant anolyte EA liquid output, and each cathode chamber produces a substantially constant catholyte EA output without requiring valving.

In another example, the anode and cathode electrodes are driven at one polarity for a specified period of time (e.g., about 5 seconds) and then driven at the reverse polarity for approximately the same period of time. If the anolyte and cathotlyte liquids are blended at the outlet of the cell, this process produces essentially one part anolyte EA liquid to one part catholyte EA liquid.

If the number of anode electrodes is different than the number of cathode electrodes, e.g., a ratio of 3:2, or if the surface area of the anode electrode is different than the surface area of the cathode electrode, then the applied voltage pattern can be used in the above-manner to produce a greater amount of either anolyte or catholyte in the produced liquid.

Referring back to FIG. 2, the applied voltage induces an electrical current across electrolysis cell 36 to generate an anolyte stream containing acidic water from the feed water flowing through anode chamber 60. This reaction also generates a catholyte stream containing an alkaline water from the feed water flowing through cathode chamber 62. The resulting anolyte stream exits anode chamber 60 through output line 90, and the catholyte stream exits cathode chamber 62 through output line 92.

In the case of a cation exchange membrane for barrier 70, upon application of a voltage potential across electrodes 72 and 74, cations originally present in the anode chamber 60 move across barrier 70 towards cathode electrode 74 while anions in anode chamber 60 move towards anode electrode 72. However, anions present in cathode chamber 62 are not able to pass through barrier 70, and therefore remain confined within cathode chamber 62.

While the electrolysis continues, the anions in the water bind to the metal atoms (e.g., platinum atoms) at anode electrode 72, and the cations in the water bind to the metal atoms (e.g., platinum atoms) at cathode electrode 74. These bound atoms diffuse around in two dimensions on the surfaces of the respective electrodes until they take part in further reactions. Other atoms and polyatomic groups may also bind similarly to the surfaces of electrodes 72 and 74, and may also subsequently undergo reactions. Molecules such as oxygen (O₂) and hydrogen (H₂) produced at the surfaces may enter small cavities in the liquid phase of the liquid (i.e., bubbles) as gases and/or may become solvated by the liquid phase of the water.

Surface tension at a gas-liquid interface is produced by the attraction between the molecules being directed away from the surfaces of electrodes 72 and 74 as the surface molecules are more attracted to the molecules within the liquid than they are to molecules of the gas at the electrode surfaces. In contrast, molecules of the bulk of the liquid are equally attracted in all directions. Thus, in order to increase the possible interaction energy, surface tension causes the molecules at the electrode surfaces to enter the bulk of the water. As a result of the electrolysis process, electrolysis cell 36 electrochemically activates the feed water by at least partially utilizing electrolysis and produces electrochemically-activated water in the form of the acidic anolyte stream (through anode chamber 60) and the basic catholyte stream (through cathode chamber 62).

Water molecules in contact with anode electrode 72 are electrochemically oxidized to oxygen (O₂) and hydrogen ions (H⁺) in the anode chamber 60, while water molecules in contact with the cathode electrode 74 are electrochemically reduced to hydrogen gas (H₂) and hydroxyl ions (OH⁻) in cathode chamber 62. The hydrogen ions in anode chamber 60 are allowed to pass through barrier 70 into cathode chamber 62 where the hydrogen ions are reduced to hydrogen gas while the oxygen gas in anode chamber 60 oxygenates the feed water to form the anolyte stream. Furthermore, since regular tap water typically includes sodium chloride and/or other chlorides, the anode electrode 72 oxidizes the chlorides present to form chlorine gas. As a result, a substantial amount of chlorine is produced and the pH of the anolyte stream becomes increasingly acidic over time.

As noted, water molecules in contact with cathode electrode 74 are electrochemically reduced to hydrogen gas and hydroxyl ions (OH⁻), while cations in the anode chamber 60 pass through barrier 70 into cathode chamber 62 when the voltage potential is applied. These cations are available to ionically associate with the hydroxyl ions produced at the cathode electrode 74, while hydrogen gas bubbles form in the liquid. Substantial amounts of hydroxyl ions accumulate over time in cathode chamber 62 and react with cations to form basic hydroxides. In addition, the hydroxides remain confined to cathode chamber 62 since barrier 70 (i.e., a cation-exchange membrane) does not allow the negatively charged hydroxyl ions pass through. Consequently, substantial amounts of hydroxides are produced in cathode chamber 62, and the pH of the catholyte stream becomes increasingly alkaline over time.

Accordingly, the electrolysis process in electrolysis cell 36 generates concentrations of reactive species and forms metastable ions and radicals in anode chamber 60 and cathode chamber 62. The electrochemical activation process typically occurs by either electron withdrawal (at anode electrode 72) or electron introduction (at cathode electrode 74), which leads to alteration of physiochemical (including structural, energetic and catalytic) properties of the feed water. It is believed that the feed water becomes activated in the immediate proximity of the electrode surfaces where the electric field intensities can reach high levels.

In the case of a barrier that is not ion selective, but has significantly larger pore sizes, such as 100 microns in diameter, or in the case of the barrier being eliminated, water (or other liquid) is introduced into the reaction(s) chamber, and a voltage potential is applied between electrodes 72 and 74. This causes water molecules in contact with or near anode electrode 72 to electrochemically oxidize to oxygen (O₂) and hydrogen ions (H⁺), while water molecules in contact or near cathode electrode 74 are electrochemically reduce to hydrogen gas (H₂) and hydroxyl ions (OH⁻). Other reactions can also occur and the particular reactions depend on the components of the water and the electrode materials. The reaction products from both electrodes 72 and 74 are able to mix and form an oxygenated fluid (for example). It has been found that the use of a barrier between the anode and cathode electrodes facilitates generation of bubbles in the output liquid. In certain embodiments, more bubbles are generated when a barrier is used than when a barrier is not used.

In addition to electrochemical activation, the electrical current that is induced through electrolysis cell 36 also heats the streams flowing through the anode and cathode chambers of the cell. This heating increases the temperatures of the resulting streams from an initial inlet temperature of the feed water to an elevated temperature, which further increases the cleaning properties of the resulting streams.

In particular, the streams are primarily heated due to the electrical resistance of the water (or other liquid) when the electrical current is induced across electrolysis cell 36 (i.e., Joule heating). Pursuant to the Joule effect, the generated heat is proportional to the electrical resistance of the water times the square of the induced electrical current, as illustrated by Equation 1:

Q˜I²×R (Equation 1)

where “Q” is the energy produced, “I” is the induced electrical current across electrolysis cell 36, and “R” is the electrical resistance of the water (or other liquid) flowing through electrolysis cell 36.

This generated heat accordingly heats the water in a manner that is based on the flow rate of the streams, the specific heat capacity of the water, and the initial temperature of the water, as illustrated by Equation 2:

Q˜M×C×(Tₒᵤₜ−Tᵢₙᵢₜᵢₐₗ) (Equation 2)

where M is proportional to the flow rate of the streams through electrolysis cell 36, “C” is the specific heat capacity of the feed water (or other liquid), “Tₒᵤₜ” is the elevated temperature of the of the resulting outlet streams through outlet lines 90 and 92, and “Tᵢₙᵢₜᵢₐₗ” is the initial temperature of the feed water entering electrolysis cell 36. Combining Equations 1 and 2 results in the relationship for heating the streams flowing through electrolysis cell 36, which is illustrated by Equation 3:

$${T}_{out} \sim \frac{{I}^{2} \times R}{M \times C} + {T}_{initial} \qquad ( Equation 3 )$$

As such, the elevated temperatures of the outlet streams from electrolysis cell 36 are proportional to the current induced through electrolysis cell 36, and inversely proportional to the flow rate of the streams through electrolysis cell 36.

Examples of suitable flow rates of the feed water into electrolysis cell 36 (or a group of cells 36) range from about 0.1 gallons/minute to about 10 gallons/minute, with particularly suitable flow rates ranging from about 3 gallons/minute to about 5 gallons/minute, depending on the number and size of the electrolysis cells, the applied voltage pattern, etc. Other flow rates may also be used.

Examples of suitable voltages applied across electrolysis cells 36 range from about 5 volts to about 60 volts, such as 50-60 volts and suitable induced electrical currents include currents of about 0.1 ampere to 10 amperes, such as about 6 amperes. As mentioned above, control electronics 32 can provide a constant DC output voltage, a pulsed or otherwise modulated DC output voltage, or a pulsed or otherwise modulated AC output voltage to electrodes 72 and 74 of electrolysis cell 36. In one embodiment, control electronics 32 may apply the voltage supplied to electrodes 72 and 74 at a relative steady state. In this embodiment, control electronics 32 and/or electrical source 42 includes a DC/DC converter that uses a pulse-width modulation (PWM) control scheme to control voltage and current output.

For example, the DC/DC converter may use a pulse of about 15 kilohertz to produce the desired voltage to electrodes 72 and 74 in the range of about 50 volts to about 60 volts. The duty cycle is dependent on desired voltage and current output. For example, the duty cycle of the DC/DC converter can be 90%. Control electronics 32 and/or electrical source 42 can also be configured, as described above, to alternate the voltage applied to electrolysis cell 36 between a relative steady state voltage at one polarity and then a relative steady state voltage at the opposite polarity for equal time periods, or different time periods to bias towards anolyte or catholyte liquids.

In the particular embodiment shown in FIG. 2, the output lines 90 and 92 from the anode chamber 60 and the cathode chamber 62 combine at 110 to form a single output line from electrolysis cell 36. This combination can be made internally or externally to cell 36. In one exemplary embodiment, all of the anolyte liquid produced in the anode chamber 60 is combined with all of the catholyte liquid produced in the cathode chamber 62 at the outlet of electrolysis cell 36 such that the cell has a single outlet 110.

As described in Field et al. U.S. Patent Publication No. 2007/0186368, it has been found that the anolyte and catholyte streams can be blended together within the distribution system of a cleaning apparatus and/or on the surface or item being cleaned while at least temporarily retaining beneficial cleaning and/or sanitizing properties. Although the anolyte and catholyte streams are blended, they are initially not in equilibrium and therefore temporarily retain their enhanced cleaning and sanitizing properties.

In a further embodiment, electrolysis cell 36 is replaced with an electrolysis cell having a single reaction chamber for the anode electrode 72 and cathode electrode 74 (i.e., no barrier 70). As such, feed line 52 and outlet line 110 directly connect to a common reaction chamber. During operation, water (or other liquid) is introduced into a common reaction chamber, and a voltage potential is applied between electrodes 72 and 74. This causes water molecules in contact with or near anode electrode 72 electrochemically oxidize to oxygen (O₂) and hydrogen ions (H⁺), while water molecules in contact or near cathode electrode 74 are electrochemically reduce to hydrogen gas (H₂) and hydroxyl ions (OH⁻). Other reactions can also occur and the particular reactions depend on the components of the water. The reaction products from both electrodes 72 and 74 are able to mix and form an oxygenated fluid (for example) since there is no physical barrier separating the reaction products from each other. As mentioned above, removing the barrier is believed to reduce the resistance between the cell electrodes and therefore increase the current applied to the liquid passing through the cell. Increasing the applied current is believed to favorably increase the amount of dissolved hydrogen and oxygen in the treated liquid.

In a further embodiment, rinse cabinet 14 has no electrolysis cells. In this embodiment, electrolysis cell(s) 36 are eliminated such that pump 34 feeds liquid directly to nozzles 38.

In a further embodiment, output lines 90 and 92 include one or more valves for selectively applying the anolyte liquid from the anode chamber 60 and/or the catholyte liquid from the cathode chamber 62 singularly, separately or in combination to nozzles 38. For example, if only one of the anolyte or catholyte liquids is supplied to the nozzles, the other of the anolyte or catholyte liquids may be dispensed to a recovery tank or to a drain of rinse cabinet 14. In another embodiment, the anolyte and catholyte liquids are fed separately to respective nozzles 38 through separate feed lines.

In the example shown in FIG. 2, the anolyte and catholyte liquids are entirely combined and fed to nozzles 38 through a liquid distribution system represented by feed lines 112.

In an exemplary embodiment, rinse cabinet 14 dispenses substantially all of the anolyte and catholyte streams upon electrical activation by electrolysis cell 36, without intermediate storage of either the anolyte stream or catholyte stream, and without feedback of any of the anolyte stream or catholyte stream into electrolysis cell 36.

Dispenser 38 may be any suitable dispenser component, such as a spray nozzle, a spigot, etc. In a particular example, each dispenser 38 include a spray nozzle, which may be selected based on a number of factors such as capacity, spray pattern, maximum and minimum pressure, etc. An example of a suitable nozzle is the 1/8HH-SS1.5Wide Full Jet Standard Spray, Small Capacity nozzle available from Spraying Systems Co. of Wheaton, Ill., USA. This nozzle has a capacity of 0.25 at 10 psi, full cone, a wide spray angle of 120 degrees at 80 psi and is made of 303 stainless steel. Other nozzles having other properties and specifications can also be used.

In an exemplary embodiment, pump 34 is operated to maintain a fluid pressure in feed lines 52 and 112 in a range of 25 psi to 60 psi, such as about 40 psi. Other fluid pressures can also be used and may vary depending on the nozzle characteristics, for example.

2.2 Spray Arrangement

As shown in FIG. 2, spray nozzles 38 may be arranged on one or both opposing sides of the carcass travel path 18 so that the nozzles dispense a liquid spray to the carcass surfaces as they pass through rinse cabinet 14. Nozzles 38 can also be positioned above or below the carcass travel path and oriented to direct a spray output to the tops and/or bottoms of the carcasses. The spray nozzles 38 can be arranged in any suitable pattern and in any suitable number. In a particular embodiment, each spray nozzle is located within a distance 114 (such as 3 inches to 6 inches) from the carcass surface (for a typically sized carcass) in order to maintain consistent contact with the spray output as the carcass passes by the nozzle and to maintain a desired electric field between the nozzle and the carcass surface, as described in more detail below.

For simplicity, FIG. 2 illustrates only one nozzle for each side of the carcass. However, any number of nozzles can be used. In a particular example, described with reference to other figures below, each side of rinse cabinet 14 may include one or more arrays of nozzles oriented to direct several output sprays to each carcass as the carcass passes the arrays of nozzles. In one more particular example, rinse cabinet 14 has a plurality of sets of nozzles on each side of the travel path. Each set contains 10 spray nozzles (or any other suitable number), which are arranged to direct 10 output sprays to a single carcass as that carcass passes the set of nozzles. When the carcass passes a midpoint of the set of nozzles along travel path 18, the output sprays of all 20 nozzles (10 on each side of the carcass) make contact with the carcass concurrently. For example, individual nozzles can be oriented to maximize the external surface area of the carcass over which concurrent contact is made. For example, in one embodiment, a sufficient number of nozzles are used and arranged such that 100% of the external surface area of each carcass is contacted concurrently, for at least a portion of travel path 18, by the output sprays of one set of nozzles on each side of the carcass.

Multiple sets (or arrays) of these nozzles can be positioned in series with one another along the travel path 18. This increases the contact time between the spray outputs and the carcasses and therefore the treatment time of each carcass within the rinse cabinet. In addition, the spacing between adjacent sets of nozzles may be set to maintain contact between at least one output spray and the carcass at all times over a predetermined length of travel path 18. This further increases the treatment time and consistency of the treatment (e.g., applied electric field) over the predetermined length of travel path 18.

As shown in FIG. 2, each nozzle 38 is fed by a respective feed line 112. In another example, a single feed line 112 may be connected to a manifold to which a plurality of distinct nozzles are mounted. Any suitable arrangement of nozzles and feed lines can be used in alternative examples. In one particular example, the nozzles 38 are spaced apart from one another in a linear direction along carcass path 18 of 5-7 inches, such as 6 inches.

Any suitable number of electrolysis cells can be used to feed any suitable number of nozzles. In a particular example, six electrolysis cells are used to feed a set of twenty nozzles, ten on each side of the carcass travel path 18.

2.3 High Voltage, Electroporation Electrode

In an illustrative embodiment of the present disclosure, an electrical charge can be delivered to the carcass through the liquid dispensed by dispensers 38 by an electrode, electrical conductor, lead, or other electrical component 40, which is separate and distinct from the electrodes in the electrolysis cells 36. The separate electrode 40 is positioned to impart, apply, or otherwise induce an electrical potential in the liquid output spray and/or stream. In the example shown in FIG. 2, electrode 40 is positioned in the liquid path to cause a separate, greater electrical potential relative to Earth ground, as compared to the potential generated by electrolysis cell 36, for example. Electrodes 40 can be located at any position along the liquid flow path from liquid source 30 to nozzles 38 (or even after nozzles 38, within the output spray paths 41) or other position as appropriate, e.g., to conduct electrical charge to the liquid dispensed by the nozzles.

In a particular embodiment, each dispenser 38 (e.g., nozzle) includes a high voltage electroporation electrode 40, which is attached to the nozzle by an electrically-conductive washer. If the nozzle 38 is electrically-conductive, the nozzle transfers the applied voltage potential to the liquid passing through the nozzle.

FIG. 4 is an exploded view of a nozzle 38 having an attached high-voltage electroporation electrode 40 according to an illustrative embodiment of the disclosure. In this example, electrode 40 is formed by a washer 150 having a terminal 152 for connecting to an electrical lead 48 (not shown in FIG. 4). Nut 154 threads onto a male end of nozzle 38, thereby securing washer 150, and thus electrode 40, in tight electrical contact with nozzle 38. An electrical lead 48 can be attached to terminal 152 for electrically connecting the terminal with control electronics 32 (shown in FIG. 2). Since washer 150 and nozzle 38 are electrically conductive, the voltage potential applied to electrode 40 is applied to the liquid flowing through the nozzle, relative to the surface being sprayed.

In another example, electrode 40 includes an adapter having two opposing ends with male connectors (e.g., barbs) for connecting between two sections of tube along one or more of the output feed lines 112, for example. The adapter has an internal lumen for passing liquid from one end to the other, along the liquid flow path of the apparatus. The adapter can be formed of or coated by any suitable material, such as an electrically-conductive material, such as copper, brass, and/or silver.

In another embodiment, electrode 40 is formed by an electrically conductive spike, which extends through a sidewall of a feed lines, such as one or more of the lines 112 such that the spike makes electrical contact with liquid flowing through the tube. Other configurations can also be used.

In another example, feed lines 112 are made at least partially of an electrically conductive material, such as a metal and/or a conductive polymer, which is electrically connected to an electrical lead 48 extending from control electronics 32. In an exemplary embodiment, the additional electrode 40 is separate from and external to electrolysis cell 36 and has no corresponding return electrode (e.g., an electrode of opposite polarity and/or an electrode representing a circuit ground for the electroporation electrode) on rinse cabinet 14. In another embodiment, rinse cabinet 14 has a ground electrode representing a circuit ground for one or more of the electrodes 40. The ground electrode can be positioned at any suitable location within rinse cabinet 14, such as in the volume of space below the carcasses, near a drain of the cabinet. It will be appreciated that other arrangements in other embodiments may be utilized.

Control electronics 32 can use the same or a different power supply as the power supply used to power electrolysis cell 36. The power supply by control electronics 32 to apply a voltage potential to control electroporation electrodes 40 can be configured to deliver an AC and/or DC voltage (such as a positive voltage) to electrodes 40 and thus to the liquid passing through nozzles 38. Various voltages and voltage patterns can be used in alternative embodiments. In embodiments in which rinse cabinet 14 does not have a dedicated ground electrode, Earth ground serves to complete the electrical circuit formed by electroporation electrode 40, the liquid streams delivered by nozzles 38, and the carcasses to which the streams are applied.

2.4 Electroporation Mechanism Example

The following discussion is provided as an example only and not intended to limit the present disclosure, operation of examples described herein and/or the scope of any issued claims appended hereto.

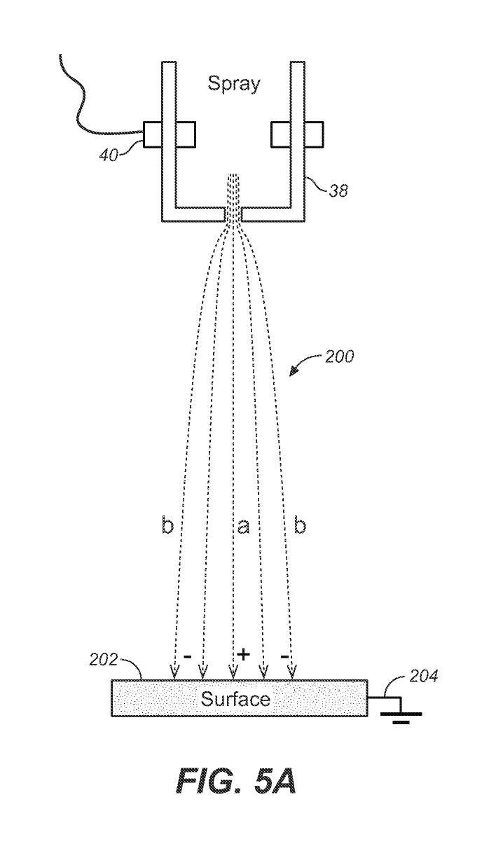

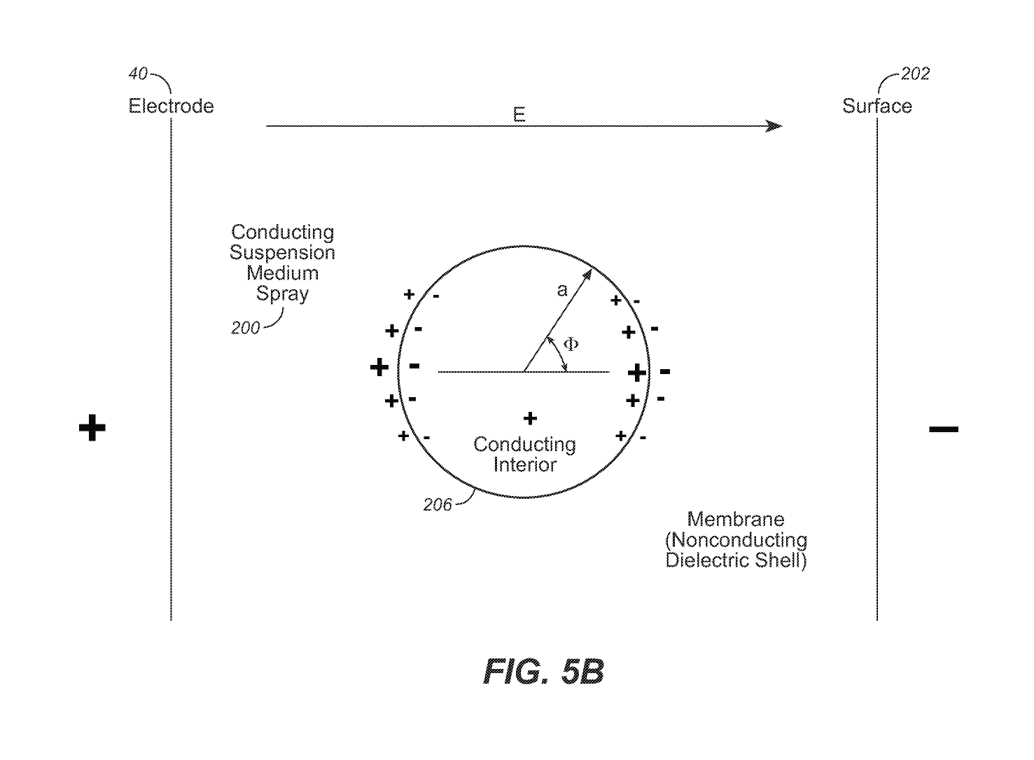

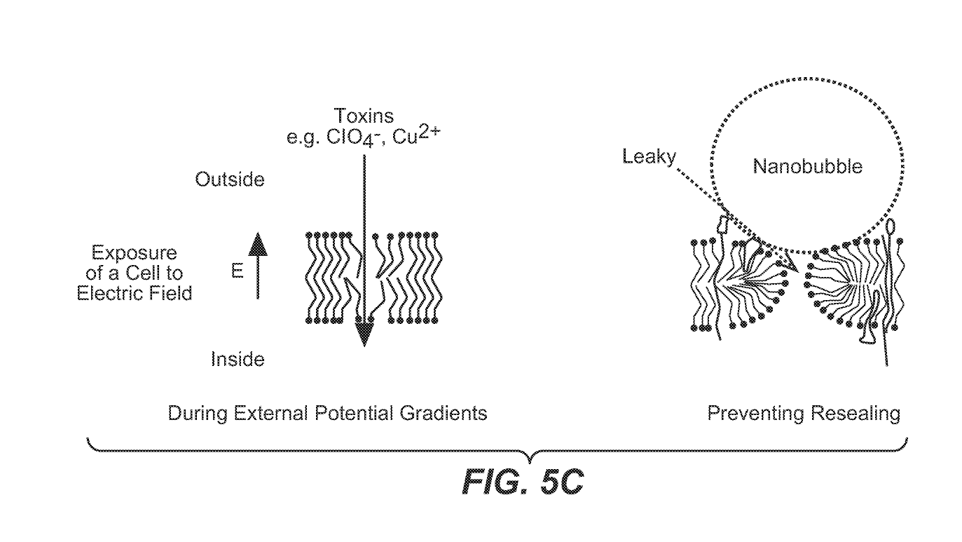

FIG. 5A is a diagram illustrating the spray output 200 from one of the spray nozzles 38, wherein individual droplets may take different paths, e.g., paths “a” and “b” from the nozzle to the surface 202 being treated. Surface 202 may or may not have an electrical conduction path to ground 204, such as Earth ground. In an example, surface 202 represents the skin of the poultry carcass passing by spray nozzle 38 by the conveyor. Nozzle 38 and surface 202 can have any relative orientation.

FIG. 5B is a diagram illustrating an example of the electroporation mechanism achieved by spraying surface 202 (in FIG. 5A) with output spray 200 from spray nozzle 38 shown in FIG. 2. The output spray 200 dispensed on surface 202 has been found to form a conducting suspension medium. FIG. 5B illustrates the resulting electric field “E” applied to a cell membrane 206 of a microorganism that is suspended from surface 202 by the dispensed liquid from output spray 200. The output spray 200 and the liquid dispensed on surface 202 together form a conductive path from electrode 40 to surface 202, for example. In one example, a measurement probe inserted just below the skin of a carcass measured an alternating voltage potential of 3 kV p-p, when the nozzle 38 was positioned 3-6 inches from the surface. Voltage potentials at the skin surface are expected, but are not required, to be in the range of approximately 2 kV to about 15 kV assuming a voltage of 7 kV to 8 kV being applied to the electrode.

The addition of an applied alternating potential from electrode 40 to the electrolytic water spray appears to endow the output spray 200 with significantly enhanced sanitizing action. This phenomenon has been associated with irreversible electroporation, for example. In one particular embodiment, the alternating potential appears to be particularly effective at about 30 kHz with a variable effect for different organisms. However, other voltage and frequencies can be used in other embodiments.

Electroporation followed by cell death is known to be achievable with a transmembrane potential of at least 0.5 V (where a membrane thickness is typically ˜3 nm, for example). In addition, the presence of cell toxins or additional mechanisms may also help prevent normally reversibly-formed pores from resealing. It should be noted that although electroporation is commonly used as a ‘reversible’ tool at lower potentials, it is recognized that, even under these conditions, often only a small percentage of cells recover.

The formation of holes in the cell membranes is generally insufficient in itself to cause cell death, as it is known that cells can survive for relatively long periods with large amounts of membrane missing.

Cell death comes because of disruption to the metabolic state of the cells, which can be caused by electrophoretic and electroosmotic (capillary electrophoretic) movement of materials into and out of the cells. Diffusion by itself is generally too slow. To achieve electrophoresis and electroosmosis, sufficient power must be dissipated within the surface, as shown in the diagram of FIG. 5C.

Different microorganisms have different total surface charges and charge distributions and therefore will react differently to each other in terms of cell death. They will also behave differently in the oscillating potential field and will have different resonant frequencies for maximum absorption (and hence maximum movement relative to the aqueous solution, causing the maximum chaos to their metabolism). Movement in and out depends primarily on potential gradients. Increased effects occur when the system is in resonance.

When considering the potential gradient delivered to the cell and the power dissipated to the sprayed surface, in one particular example, the spray device delivers a fine spray that may be partially a true aerosol (˜1μ droplets), but mostly a mist with droplet sizes much greater than 10μ. The droplet sizes and velocity profiles can vary between different embodiments.

The velocity of the liquid exiting the nozzle is simply calculated from the rate of liquid sprayed divided by the area of the exit orifice. However the subsequent decrease in droplet speed depends on the droplet size (mass to surface area ratio). The terminal velocity of 10μ and 50μ droplets are only about 10⁻³ m/s and 10⁻¹ m/s respectively.

Sprayed water droplets descend at different rates, and the time differences will be significant when related to the rapidly alternating potential (e.g., 28 kHz). For example, in FIG. 5A, pathway (b) will be longer than pathway (a), for example by about 1 cm. The descent velocity (dependent on the drop size, flow rate and nozzle diameter) will determine the difference in time between the drops landing but this is likely to be several to many times the potential cycling time of 36 μs, for example.

Cells with open pores are much more prone to the effects of cell toxins in the aqueous solution as they have no barrier to their entry. The potential cell toxins co-delivered with the alternating potential are peroxide, chlorine oxides, and other redox agents such as superoxide, ozone and singlet oxygen, and heavy metal ions such as cupric ions and/or silver ions.

Charged nanobubbles will move in the electric fields and will be capable of picking up materials from the surface. As they are surface-active, they may additionally interfere with pore resealing and preferentially deliver their cytotoxic surface active molecules to the pore sites, as shown in FIG. 5C, for example.

In view of the above, the electrolyzed water produced by the electrolysis cell 36, shown in FIG. 2, for example, acts as a cleaning agent due to production of tiny electrically-charged bubbles. These attach themselves to dirt particles/microorganisms and transfer their charge. The charged and coated particles separate one from another due to the repulsion between their similar charges and enter the solution as a suspension. Coating of the dirt by tiny bubbles promotes their pick-up by larger buoyant bubbles that are introduced during cleaning, thus aiding the cleaning process. Simultaneously, microorganisms can be electroporated and killed or otherwise eliminated by the electric potential generated by the additional electrode 40, thereby reducing the number of live microorganisms on a surface.

Thus, to enhance sanitization ability properties, electroporation can be used for example to accomplish a more consistent and effective destruction of microbial action by discharging (in a relative sense) a high-voltage to a ground (such as Earth ground) through an aqueous fluid. In some embodiments, the livestock carcass itself serves as an Earth ground since it has a different charge level than the high voltage electrode. In addition, the carcass may provide an electrical path to ground through the shackle and conveyor materials.

It has also been found that the combination of the electrochemically-activated liquid produced by the electrolysis cell and the electric field applied by the electroporation electrode has a synergistic effect. It is believed that as the charged nanobubbles produced in the electrochemically-activated liquid move in the electric fields, they pick up microorganisms and separate them from the surface. By separating the microorganisms from the surface, such that they are suspended in the liquid on the surface, the electric field produced along the surface by the electroporation electrode is applied more easily across the microorganism cells. Whereas, if the microorganism is in contact with the surface, the electric field is more easily discharged into the surface ground and may be less effective in creating irreversible electroporation of the organisms cells. With the cell suspended, the applied alternating field oscillates back and forth causing damage to the cells.

In addition, it has been found that the electric field produced through the output spray within the rinse cabinet 14 by the electroporation electrode is effective in killing microorganism cells present in the mist environment surrounding the carcasses as the carcasses are cleaned and sanitized by the spray outputs, which reduces contamination within the rinse cabinet itself and within the processing plant as a whole.

2.5 Example Electroporation Voltage Waveform

As mentioned above, control electronics 32 apply a voltage potential to electroporation electrodes 40, which can be configured to deliver an AC and/or DC voltage (such as a positive voltage) to electrodes 40 and thus to the liquid passing through nozzles 38.

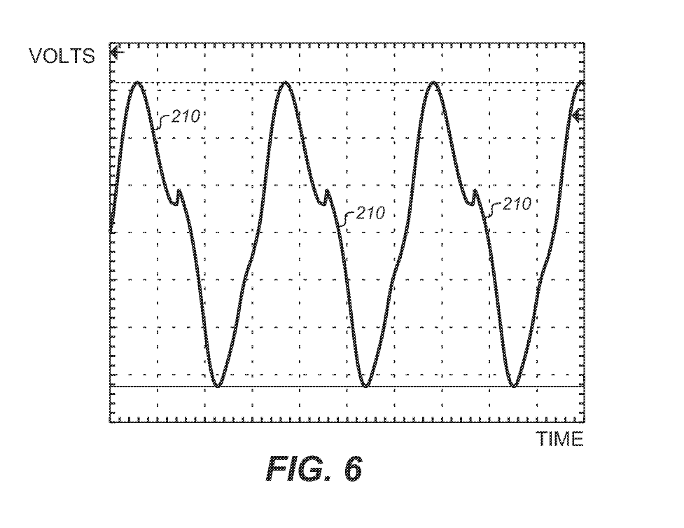

FIG. 6 is a waveform diagram illustrating the voltage pattern applied to each electroporation electrode 40 in one particular example. In this example, the shape of the waveform is a combination of a sine wave and a square wave. However, the waveform can have other shapes, such as a sine wave, a square wave, or other waveform. In a particular example, the applied voltage has an AC voltage of 2 kV to 20 kV peak-to-peak, for example, when liquid is flowing through nozzles 38 and has a frequency of about 30 kHz. Other voltages and frequencies can also be used. In this example, the frequency remains substantially constant as the apparatus (e.g., rinse cabinet 14) dispenses electrochemically-activated liquid to the livestock carcasses being treated. In another example, the frequency is maintained in a range of about 20 kHz and 100 kHz, between 25 kHz and 50 kHz, and between 28 kHz and 46 kHz. The current between the nozzle and carcass is relatively small, such as between zero to 100 milliamps, for example.

In the example shown in FIG. 6, the control electronics 32 are configured to generate and apply to the electroporation electrode a voltage having a sinusoidal waveform comprising at least one step 210 on an edge of the waveform, wherein each step comprises a local peak, and wherein the electroporation electrode is arranged and positioned within rinse apparatus 14 to generate an alternating electric field between the electrode and each carcass passing by the electrode, in response to the applied voltage. The inventors of the present application have found that such a discontinuous waveform improves the killing or deactivation of microorganisms achieved through the resulting output spray that is applied to the carcasses.

In another example, the frequency varies over a predefined range while the apparatus dispenses electrochemically-activated liquid to the carcasses being treated. For example, the control circuit that drives electroporation electrode 40 can sweep the frequency within a range between a lower frequency limit and an upper frequency limit, such as between 20 kHz and 100 kHz, between 25 kHz and 50 kHz, and between 30 kHz and 60 kHz. The frequency can have any suitable waveform over time, such as a triangular or sawtooth waveform, from a low frequency limit to a high frequency limit and then back down to the low frequency limit over a period of about 0.1 second to about 10 seconds, for example. Since different microorganisms might be susceptible to irreversible electroporation at different frequencies, the killing effect of the applied voltage is swept between different frequencies to potentially increase effectiveness on different microorganisms. For example, sweeping the frequency might be effective in applying the potential at different resonant frequencies of different microorganisms.

The following sections describe exemplary control circuit for driving the electrolysis cells 38 and the electroporation electrodes 40 within the systems shown in FIGS. 1 and 2, for example.

2.6 Example Control Circuit for Electrolysis Cells

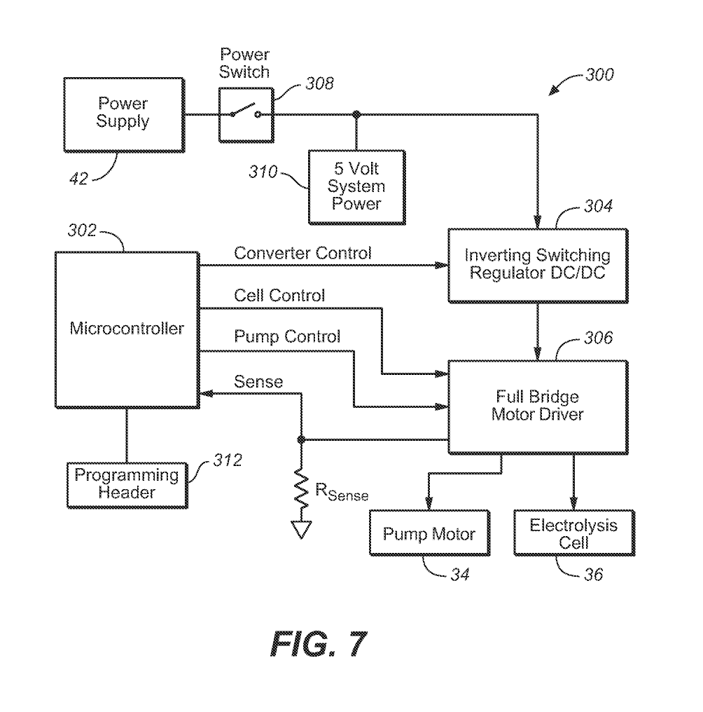

FIG. 7 is a block diagram of an example of a control circuit 300 within control electronics 32 (shown in FIG. 2) for controlling the electrolysis cell(s) according to an exemplary aspect of the disclosure. The main components of control circuit 300 include a microcontroller 302, a DC-to-DC converter 304, and an output driver circuit 306.

Power to the various components is supplied by power supply 42. A power switch or other control component 308 an output voltage to voltage regulator 310 and to DC-to-DC converter 304. Any suitable voltage regulator can be used, such as an LM7805 regulator from Fairchild Semiconductor Corporation. In a particular example, voltage regulator 310 provides a 5 volt output voltage for powering the various electrical components within the control circuit.

DC-to-DC converter 304 generates an output voltage to be applied across the electrodes of electrolysis cell(s) 36. The converter is controlled by microcontroller 302 to step the drive voltage up or down in order to achieve a desired current draw through the electrolysis cell. In a particular example, converter 304 steps the voltage up or down between a range of 8 volts to 60 volts, such as between 50 volts and 60 volts, (or greater) to achieve a current draw through electrolysis cell(s) 36 of about 6 amps, as pump 34 pumps water through cell(s) 36 and out nozzle(s) 38 (FIG. 2) at a rate of 4 gallons per minute, for example. The required voltage depends in part on the conductivity of the water between the cell's electrodes and the geometry of the electrolysis cells. Other voltages, currents and liquid flow rates can be used in other examples.

In a particular example, DC-to-DC converter 304 includes a Series A/SM surface mount converter from PICO Electronics, Inc. of Pelham, N.Y., U.S.A. In another example, converter 1004 includes an NCP3064 1.5A Step-Up/Down/Inverting Switching regulator from ON Semiconductor of Phoenix, Ariz., U.S.A, connected in a boost application. Other circuits and/or arrangements can be used in alternative embodiments.

Output driver circuit 306 selectively reverses the polarity of the driving voltage applied to electrolysis cell(s) 36 as a function of a control signal generated by microcontroller 302. For example, microcontroller 302 can be configured to alternate polarity in a predetermined pattern, such that shown and/or described with reference to FIG. 3. Output driver 306 can also provide an output voltage to pump 34. Alternatively, for example, pump 34 can receive its output voltage directly from the output of switch 308, for example.

In a particular example, output driver circuit 306 includes a DRV 8800 full bridge motor driver circuit available from Texas Instruments Corporation of Dallas, Tex., U.S.A. Other circuits and/or arrangements can be used in alternative embodiments. The driver circuit 306 has an H-switch inverter that drives the output voltage to electrolysis cell(s) 36 according to the voltage pattern controlled by the microcontroller. The H-switch also has a current sense output that can be used by the microcontroller to sense the current drawn by cell 36. Sense resistor R_{SENSE} develops a voltage that is representative of the sensed current and is applied as a feedback voltage to microcontroller 302. Microcontroller 302 monitors the feedback voltage and controls converter 304 to output a suitable drive voltage to maintain a desired current draw.

Microcontroller 302 also monitors the feedback voltage to verify that electrolysis cell(s) 36 and/or pump 34 is operating properly. Microcontroller 302 can include any suitable controller, processor, and/or circuitry. In a particular embodiment, it includes an MC9S08SH4CTG-ND Microcontroller available from Digi-Key Corporation of Thief River Falls, Minn., U.S.A.

The control circuit 300 further includes a control header 312, which provides an input for programming microcontroller 302.

In one particular example, the elements 302, 304, 306, 308, 310 reside on circuit board.

2.7 Example Control Circuit for Electroporation Electrodes(s)

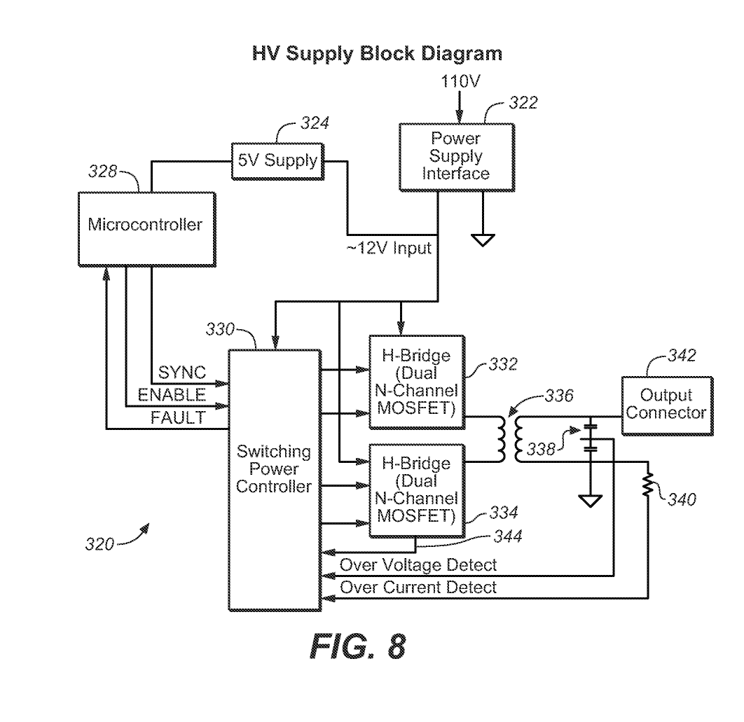

FIG. 8 is a block diagram of an example of a control circuit 320 within control electronics 32 (shown in FIG. 2) for controlling the electroporation electrode(s) 40 according to an exemplary aspect of the disclosure.

Circuit 320 includes a power supply interface 322, voltage regulator 324, microcontroller 328, switching power controller 330, H-bridge circuits 332 and 334, transformer 336, voltage divider 338, sense resistor 340 and output connector 342.

Input connector 322 receives a supply voltage from a main circuit board, such as that shown in FIG. 7 for example, and supplies the voltage to voltage regulator 324, switching power controller 330 and H-bridge circuits 332 and 334. In a particular example, voltage regulator 324 provides a 5 volt output voltage for powering the various electrical components within the control circuit 320, such as microcontroller 328 and switching power controller 330. Any suitable voltage regulator can be used, such as an LM7805 regulator from Fairchild Semiconductor Corporation.

In this embodiment microcontroller 328 provides a clock signal (SYNC) and an enable signal (ENABLE) to switching power regulator 330, and monitors for fault conditions. In one example, microcontroller 328 comprises an ATtiny24 QPN Microcontroller available from ATMEL Corporation. Other controllers can be used in alternative embodiments.

The clock signal SYNC provides a reference frequency for switching power controller 330. Enable signal ENABLE, when active, enables (or turns on) switching power controller 330. Normally, microcontroller 328 sets ENABLE to an active state and monitors the FAULT signal for a fault condition. When controller 330 indicates a fault condition by activating the signal FAULT, microcontroller 328, selectively pulses the ENABLE signal to an inactive state and then returns it to the active state to reset switching power controller 330. If the fault condition clears, microcontroller continues to operate switching power controller normally. If the fault condition remains active, then microcontroller 328 activates a fault indicator (not shown).

In one example, switching power controller 330 includes a TPS68000 CCFL Phase Shift Full Bridge CCFL Controller available from Texas Instruments. However, other types of controllers can be used in alternative embodiments.

Based on the SYNC signal, switching power controller 330 provides gate control signals to the gates of switching transistors within the H-bridge circuits 332 and 334. In one example, H-bridge circuits 332 and 334 each include an FDC6561AN Dual N-Channel Logic Level MOSFET (although other circuits can be used), which are connected together to form an H-bridge inverter that drives the primary side of transformer 336 with the desired voltage pattern, such as that shown in FIG. 6. Transformer 336 steps the drive voltage from about 10V-13V peak-to-peak up to about 2 kV to 20 kV, for example, when liquid is being dispensed from the apparatus. The output drive voltage is applied to the electroporation electrode 40 through output connector 342.

Voltage divider 338 comprises a pair of capacitors that are connected in series between the primary side of the transformer and ground to develop a voltage that is feed back to switching power controller 330 and represents the voltage developed on the secondary side of the transformer. This voltage level is used to detect an over-voltage condition. If the feedback voltage exceeds a given threshold, switching power controller 330 will activate fault signal FAULT.

Sense resistor 340 is connected between the primary side of the transformer and ground to develop a further feedback voltage that is feed back to switching power controller 330 and represents the current flowing through the secondary side of the transformer. This voltage level is used to detect an over-current condition. If the feedback voltage exceeds a given threshold, switching power controller 330 will activate fault signal FAULT, indicating a fault in the transformer.

In addition, the source of the bottom transistor in one leg of the H-bridge is fed back to switching power controller 330, as shown by arrow 344. This feedback line can be monitored to measure the current in the primary side of the transformer, which can represent the current delivered to the load through electroporation electrode 40. Again, this current can be compared against a high and/or a low threshold level. The result of the comparison can be used to set the state of fault signal FAULT.

2.8 Exemplary Electrolysis Cell

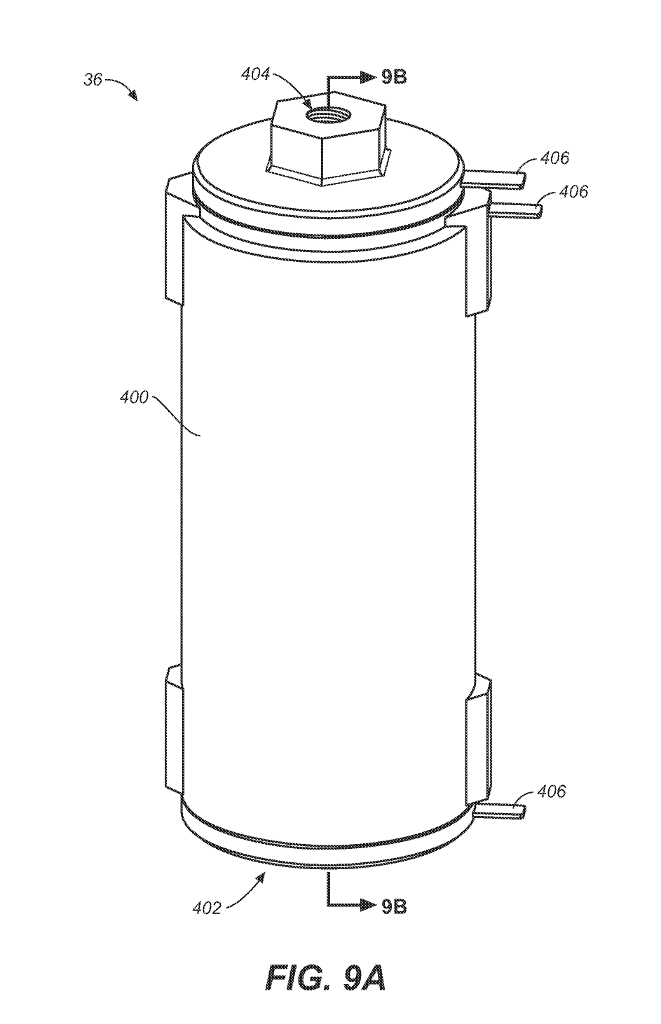

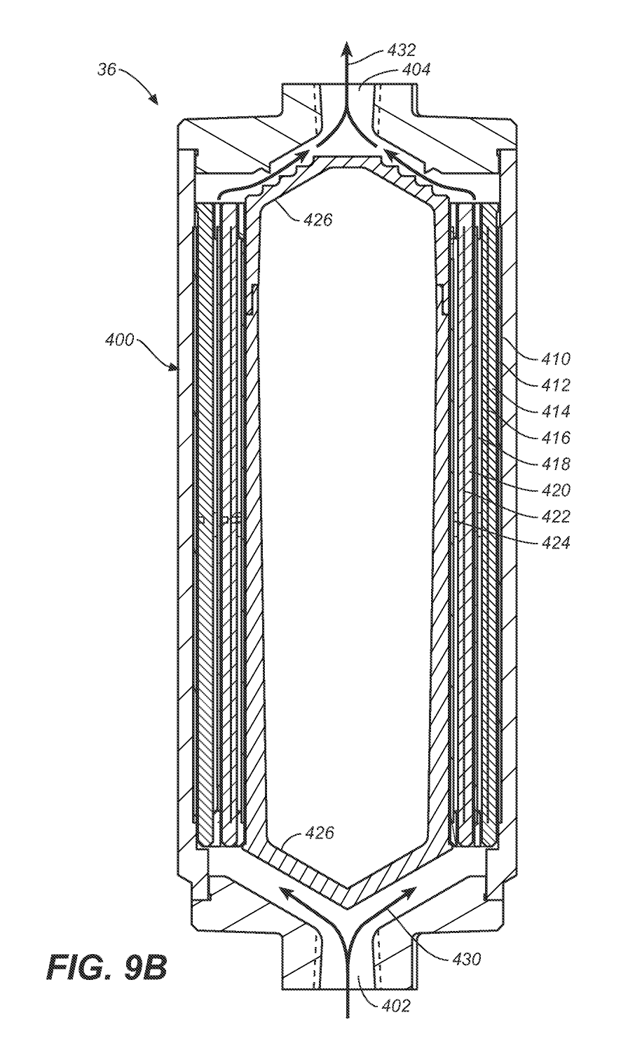

FIG. 9A is a perspective view of an electrolysis cell 36 according to an exemplary aspect of the disclosure, which can be used in the rinse cabinet shown in FIGS. 1 and 2.

In this non-limiting example, electrolysis cell 36 has a cylindrical shape with a housing 350, an inlet 402, and outlet 404, and electrical terminals 406. Fluid from feed lines 52 (shown in FIG. 2) enters inlet 402 and exits outlet 404. Outlet 404 can be coupled to one or more of the outlet feed lines 112, shown in FIG. 2. In this example, electrolysis cell has three cylindrical electrodes arranged coaxially with one another, each of which is electrically coupled to a respective terminal 406. Depending on the relative polarity of voltages applied to the terminals 406, the electrolysis cell may include two anode electrodes surrounding a single cathode electrode or may include two cathode electrodes surrounding a single anode electrode. Many other arrangements and numbers of electrodes are also possible.

FIG. 9B is a cross-sectional view of the electrolysis cell 36 taken along lines 9B-9B of FIG. 9A. Within cylindrical housing 400, cell 36 includes a liner (such as polyprolylene) 410, a first, outer electrode 412, a gap 414 containing a first, outer barrier 416, a second, middle electrode 418, a gap 420 containing a second, inner barrier 422, and an inner electrode 424. The first gap 414 is positioned between outer electrode 412 and middle electrode 418, and contains the first barrier 416. The second gap 420 is positioned between middle electrode 418 and inner electrode 424, and contains the second barrier 422.

An inner core 426 blocks liquid from passing through the center of cell 36, and diverts liquid entering inlet 402 along the direction of arrows 430. This liquid enters the gaps 414 and 420 between the electrodes and passes along the electrodes 412, 418, and 424, on either side of the barriers 416 and 422. The liquid then exits outlet 404 along arrows 432. Anolyte liquid produced in the anode chamber, formed between the anode electrode and a respective barrier, and catholyte liquid produced in the cathode chamber, formed between the cathode electrode and a respective barrier, blend together as the liquid exits single outlet 404.

In a particular example, electrodes 412, 418 and 424 are made of a titanium mesh coated with iridium oxide, which are spaced apart from one another by a gap of about 0.030 inches (0.76 mm). The barriers 416 and 422 are constructed of polypropylene sheets having a thickness of 10 mils (0.254 mm).

As mentioned above, the barriers 416 and 422 can be removed in an alternative embodiment.

3. Prototype Rinse Cabinet

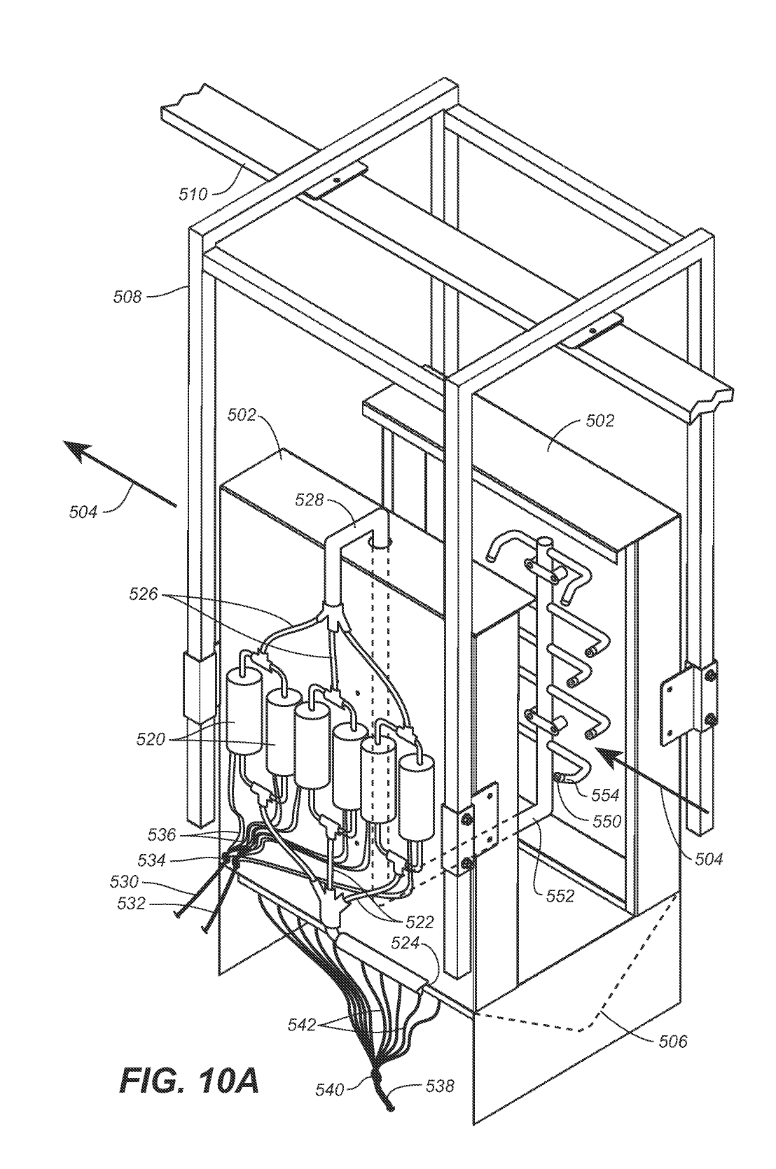

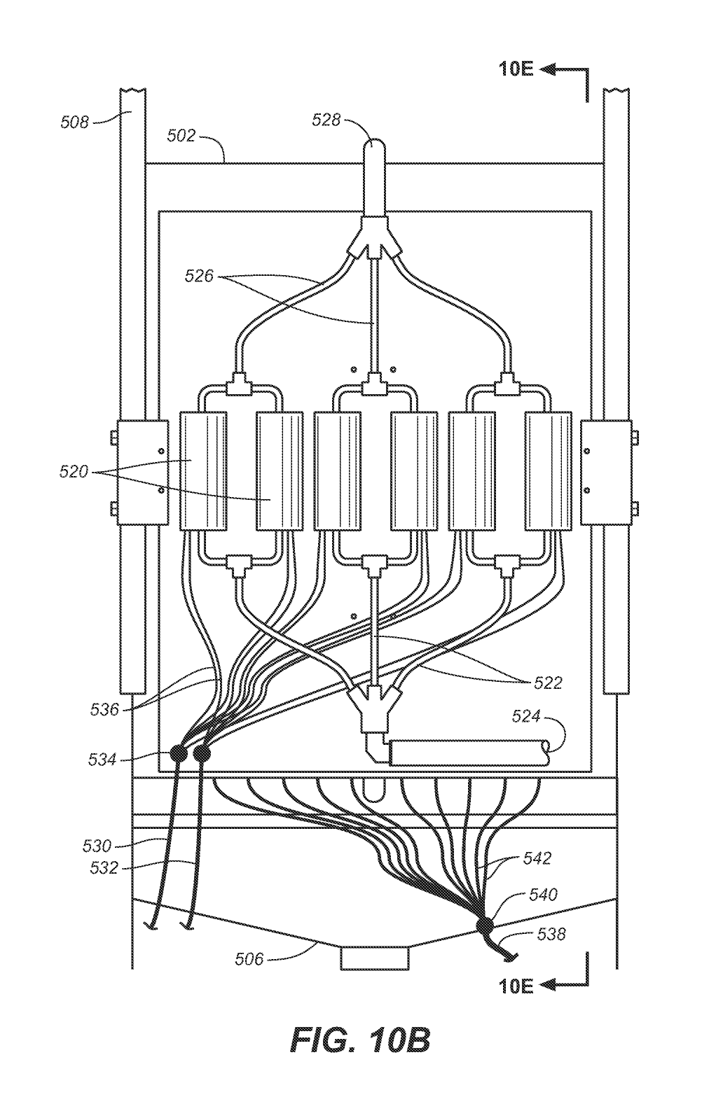

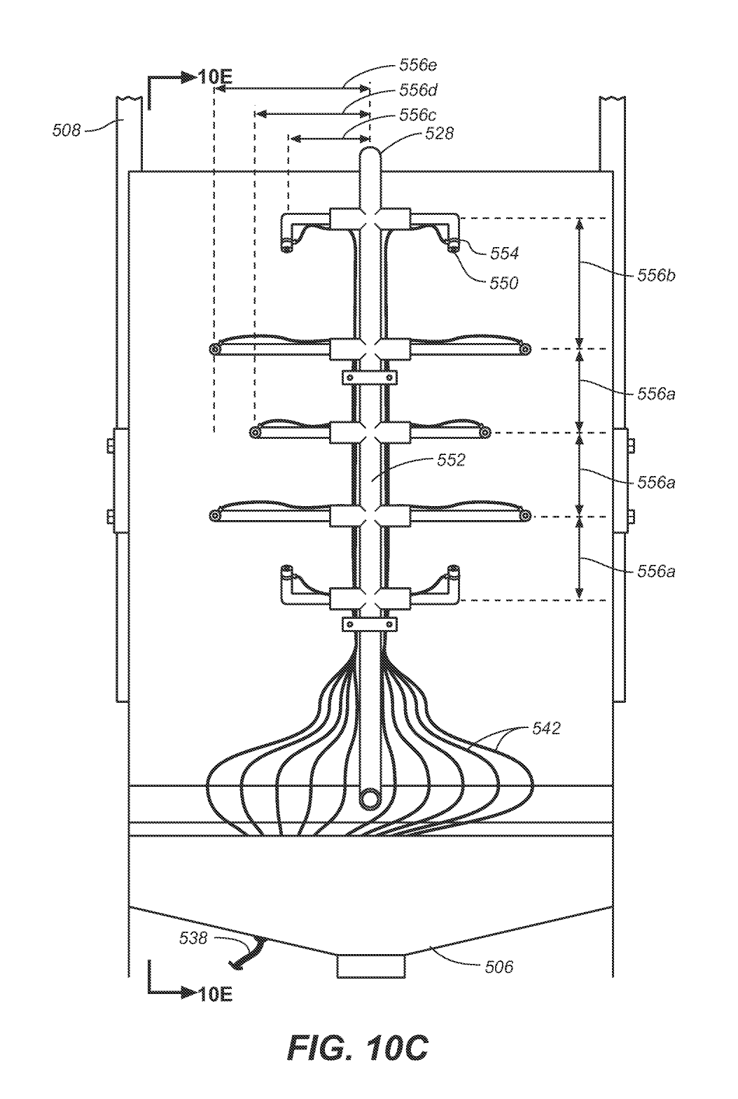

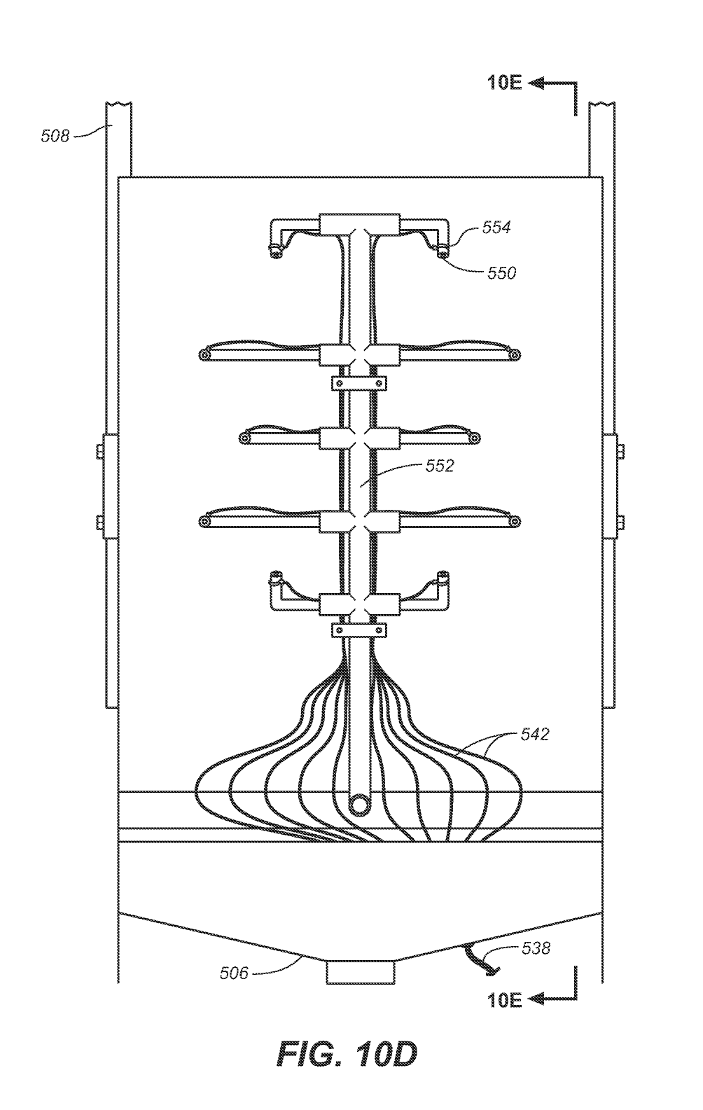

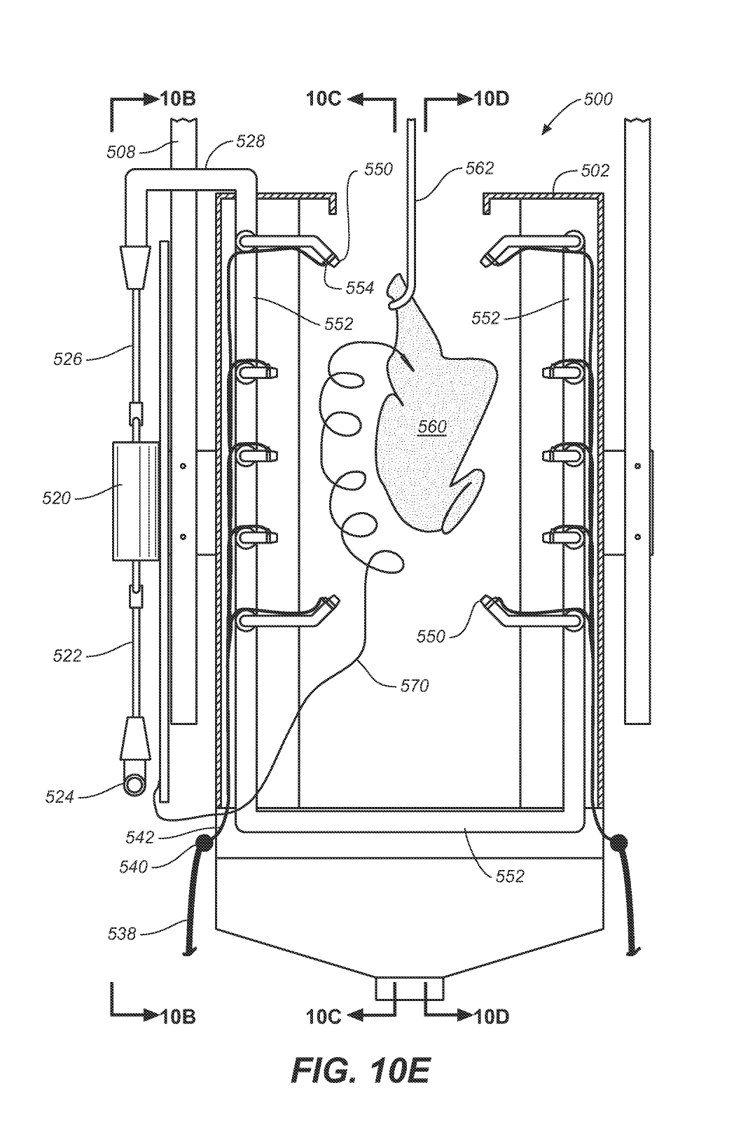

FIG. 10A is a perspective view of a prototype rinse cabinet 500 according to an exemplary aspect of the present disclosure. Rinse cabinet 500 includes a housing 502, forming a partial enclosure about a carcass travel path 504. Housing 502 defines first and second opposing sides of the rinse cabinet 500 relative to travel path 504. Housing 502 has a base 506, which forms a drain pan for collecting liquid sprayed onto the carcasses. A frame 508 is attached to the housing for supporting an overhead conveyor 510. Conveyor 510 is configured to carry one or more poultry shackles (shown in FIG. 10D) similar to those shown in FIG. 1 along the travel path 504.

As shown in FIGS. 10A and 10B, six electrolysis cells 520 are mounted to one side of housing 502. Electrolysis cells 520 are similar to the electrolysis cell described with reference to FIGS. 9A and 9B. In this example, cells 520 have electrodes made of a titanium mesh coated with iridium oxide, which are spaced apart from one another by a gap of about 0.030 inches (0.76 mm), and have barriers constructed of polypropylene sheets having a thickness of 10 mils (0.254 mm).

Cells 520 are electrically and fluidically coupled together in parallel with one another and are fed by feed lines 522, which receive a feed liquid from inlet 524 and distribute the feed liquid to the inlet of each cell 520. Feed lines 522 are formed by ½ inch PVC pipe or flexible tubing, for example. Inlet 524 can be coupled to a liquid source, such as a source of regular tap water, for example. The outlets of cells 520 are coupled to a set of outlet feed lines 526, which merge together to form a single outlet feed line 528. Rinse cabinet 500 also includes a pump (not shown) for pumping the feed liquid through the feed lines 522 at the desired rate and pressure.

A pair of electrical cables 530 and 532 is connected to terminal blocks 534 for supplying electrical power to the electrolysis cells 520, provided by a power supply (not shown). Electrical cables 530 and 532 are driven by a control circuit (not shown), such as those shown and described with reference to FIGS. 2 and 7. However in one or more tests, the power supply included a conventional test bench power supply that delivered constant DC voltage to the cells. A plurality of electrical cables 536 are connected between terminal blocks 534 and respective terminals of the electrolysis cells 520 (such as terminals 406 shown in FIG. 9A). A suitable number of cables 536 can be used depending on the number of terminals 406 and their electrical configuration.

Rinse cabinet 500 further includes a high voltage electroporation input cable 538, which is connected to a high voltage control circuit (not shown), similar to those shown and discussed with respect to FIGS. 2 and 8. High voltage input cable 538 is connected to a terminal block 540, which distributes an applied voltage to the electroporation electrodes of each spray nozzle through a plurality of respective electrical cables 542. Each cable 542 is electrically connected to a respective nozzle in rinse cabinet 500. For simplicity, only ten cables 542 are shown in FIG. 10A. In this particular prototype, the electrical cables 538 and 542 are similar to standard automobile spark plug wires.

Outlet feed line 528 feeds electrolyzed liquid to the plurality of nozzles contained in rinse cabinet 500. Each side of rinse cabinet 500 contains a respective array of spray nozzles 550 directed toward the travel path 504, although only one set of nozzles 550 is visible in FIG. 10A. Nozzles 550 are coupled to feed lines 552, which are fluidically coupled to outlet feed line 528. As shown in more detail in FIGS. 10C and 10D, in this particular prototype, each side of rinse cabinet 500 includes an array of ten nozzles 550 oriented to direct an output spray onto a carcass travelling along travel path 504. FIG. 10C is a partial elevation view of a first side of the prototype rinse cabinet shown in FIG. 10A. FIG. 10D is a partial elevation view of a second, opposing side of the prototype rinse cabinet shown in FIG. 10A.

Referring to FIG. 10C, the nozzles are separated vertically and horizontally from one another relative to the travel path. The middle six nozzles 550 as viewed in the vertical direction, are oriented essentially normal to travel path 504, and the upper two and lower two nozzles 550 are oriented slightly downward and slightly upward, respectively, in order to direct output sprays toward the top and bottom of the carcass as the carcass passes the spray nozzles. These nozzles are also oriented slightly inward toward one another in order to better contact the leading and trailing surfaces of the carcass as the carcass travels along path 504. As shown in FIG. 10D, a similar array of nozzles 550 is positioned on the other side of housing 502, which oppose the nozzles shown in FIG. 10C relative to travel path 504. Thus, the nozzles 550 in rinse cabinet 500 are positioned to maintain direct and consistent contact between the various spray outputs and substantially the entire external surface of the carcass as the conveyor moves the carcass along the travel path.

In the example shown in FIGS. 10C and 10D, each array includes five rows of nozzles 550 that are vertically separated from one another, with two nozzles in each row. The nozzles in the bottom four rows are separated vertically from one another by a distance 556a of about 4 inches, center-to-center. The top, fifth row is separated vertically from the fourth row by a distance 556b of about 6 inches, center-to-center.

The nozzles in the first, bottom row and the top, fifth row are separated horizontally from the center of the vertical feed line 552 by a distance 556c of about 3 inches. The nozzles in the second row and the fourth row are separated horizontally from the center of the vertical feed line 552 by a distance 556d of about 6 inches. The nozzles in the third row from the bottom are separated horizontally from the center of the vertical feed line 552 by a distance 556e of about 4 inches. The nozzles in the fourth row are separated horizontally from the center of the vertical feed line 552 by about 6 inches. The nozzles in the fifth, top row are separated horizontally from the center of the vertical feed line 552 by about 3 inches.

As shown in FIG. 10E, the nozzles in the first, bottom row extend out toward the travel path by about 4 inches. The nozzles in the second, third and fourth rows extend out toward the travel path by about 1 inch. The nozzles in the fifth rows extend out toward the travel path by about 4 inches.

The above-arrangements and spacings are provided as examples only. Other numbers and arrangements of nozzles can be used in other embodiments.

As mentioned above, in a commercial embodiment, the rinse cabinet might have a longer carcass travel path and may include further arrays (and/or larger arrays) of nozzles positioned adjacent to one another along the travel path. For example, adjacent arrays may be positioned such that the outer-most nozzles of one array are spaced from the outer-most nozzles of the next, adjacent array along the travel path by a distance of about 3 inches to 12 inches, such as a distance of 6 inches. Other separation distances can also be used.

As also shown in FIG. 10C-10E, each nozzle 550 includes an electroporation electrode 554 connected to the nozzle. The electrodes 554 and nozzles 550 are similar to those shown in FIG. 4. Each electrode 554 is connected to the high voltage electroporation input cable 538 through a respective electrical cable 542. As mentioned above, rinse cabinet 500 has no corresponding return electrode(s) (e.g., an electrode of opposite polarity and/or an electrode representing a circuit ground) for the electroporation electrodes 554. In another embodiment, rinse cabinet 500 has a ground electrode representing a circuit ground for one or more of the electrodes 554. The ground electrode can be positioned at any suitable location within rinse cabinet 500 within the spray environment, such as on one of the central, vertical feed lines 552 or in the volume of space below the carcasses, near a drain of the cabinet. The ground electrode can be coupled to the control circuit 320 shown in FIG. 8 or to earth ground, for example. It will be appreciated that other arrangements in other embodiments may be utilized.

FIG. 10E is an end elevation view of the prototype rinse cabinet 500 shown in FIGS. 10A-10D. In this figure, the two sets of spray nozzles 550 are visible on opposing sides of the carcass travel path 504 (which is into the page in FIG. 10E). A carcass 560 is illustrated hanging from a shackle 562 between the two sets of spray nozzles 550. Each of the spray nozzles are positioned within 3 inches to 6 inches from the carcass 560. Also shown in FIG. 10E is a measurement electrode 570, which was inserted just below the skin of the carcass 560. In one test, when a voltage of about 7 kV was applied to electrodes 550, measurement electrode 570 measured a voltage of 3.5 kV just under the skin surface. It is estimated that when a voltage of 7 kv to 8 kV is applied to electrodes 550, the electric field at the skin surface is about 5 kV. In one embodiment, the power supply and control circuit are configured to apply a voltage of 2 kV to 20 kV peak-to-peak to the electrodes 550, such that the voltage at the skin surface is about 2 kV to about 15 kV.

As described above, rinse cabinet 500 applies an output spray from each nozzle 550, which has enhanced cleaning and sanitizing properties, to the carcass. The cleaning properties are enhanced, for example, by electrolysis cells 520, which produce tiny electrically-charged bubbles in the liquid sprayed onto the carcass surface. These bubbles attach themselves to dirt particles/microorganisms and transfer their charge. The charged and coated particles separate one from another due to the repulsion between their similar charges and enter the solution as a suspension. Coating of the dirt by tiny bubbles promotes their pick-up by larger buoyant bubbles that are introduced during cleaning, thus aiding the cleaning process. The suspended dirt particles and microorganisms are mechanically removed from the carcass by the rinsing action provided by the spray outputs.

Simultaneously, microorganisms on the carcass surface or suspended from the carcass surface can be electroporated and killed or otherwise eliminated by the alternating electric field generated by the electroporation electrodes 554, thereby reducing the number of live microorganisms on a surface. Thus, the sanitizing properties of the output spray are enhanced, for example, by applying the electric field to the surface of the carcass through the output spray. The electric field applied to the carcass, and thus to cells of microorganisms on a carcass, meets or surpasses a threshold such that the cells become permanently damaged by a process known as irreversible electroporation, for example. If the electric field threshold is reached or surpassed, electroporation will compromise viability of the cells, resulting in irreversible electroporation. Thus, rinse cabinet 500 is configured to deliver an applied electric field to the carcass through the charged output spray, which exceeds the electric field threshold.

The microorganisms are suspended from the surface of the carcass by the liquid dispensed from spray nozzles and through which the electric field is applied. Other mechanisms, such as surfactant additives can also be used to alter the liquid's oxidation reduction potential and/or enhance the suspension of particles and microorganisms from the carcass surface.

3.1 Test Results

The prototype rinse cabinet 500 shown in FIGS. 10A-10D was used to test the efficacy of the rinse cabinet in reducing bacterial counts on test surfaces treated within the rinse cabinet.

In the following tests, the electrolysis cells were operated at 50V-60V such that each cell drew about 6 amperes between the electrodes. Regular tap water was pumped through the feed lines at a pressure of about 40 psi, where the flow rate through the combined six cells was about 4 gallons per minute. The high voltage, electroporation electrodes 554 were driven with a sinusoidal voltage waveform from a test bench power supply amplitudes of about 2 kV peak-to-peak and 4 kV peak-to-peak and a frequency of about 30 kHZ, as explained below.

3.1.1 Test Vehicle

For each test, a test vehicle was prepared by inoculating 1″×1″ pieces of VITRO-SKIN® with bacteria and attaching the piece to the external surface of a small 6-inch to 8-inch section of PVC pipe, which was capped at both ends. The test vehicle was then hung from a shackle attached to the conveyor 510 (shown in FIG. 10A) such that the test vehicle would pass between the plurality of spray nozzles 550 in rinse cabinet 500.

The control circuits that drive electrolysis cells 520, electroporation electrodes 554 and the pump were then activated so that spray nozzles 550 delivered output sprays of electrolyzed water to the travel path within the cabinet, which conducted the applied electric fields from the respective electroporation electrodes 554.

The conveyor 510 was then activated to move the test vehicle along travel path 504, through the rinse cabinet 500, at a desired rate. Once the test vehicle had passed through the rinse cabinet 500, the pieces of VITRO-SKIN® were removed from the test vehicle so that remaining bacterial colonies could be counted.

3.1.2 Materials

The materials for each test included:

S. enterica ATCC 10708 culture (overnight)

Trypticase soy broth (TSB)

Trypticase soy agar plates (TSA)

VITRO-SKIN® (hydrated overnight)

Sterile buffered peptone water (BPW)

Sterile forceps

125 mm×16 mm sterile tubes

Pipettors, tips

3×5″ sterile stomacher bags

Sterile 5 mL pipette

Vortex mixer

T-pins

Pipet aid

Micropipettors

Yellow micropipette tips

Blue micropipette tips

3.1.3 Test Method

### Day 1

1. Cut 1″×1″ pieces of VITRO-SKIN®
2. Dilute 52 g glycerol into 298 g water, mix, and place in the bottom of the Red Lid Hydration Chamber (IMS, Inc.).
3. Place pieces of VITRO-SKIN® above the liquid on the tray provided, seal the lid and allow the VITRO-SKIN® to hydrate for 16-24 hours, but not over 24 hours.
4. Inoculate a culture of S. enterica grown in trypticase soy broth (TSB) to be used the next day (18-24 hours).

### Day 2 (Test Day)

5. Dilute culture 1:5 in buffered peptone water (BPW)˜[10⁷ CFU/mL] to make the inoculum.
6. Dilute the inoculum 1:10 in BPW and plate 20 μL of dilutions 4-6 on TSA plates.
7. Using a P20 micropipettor, pipette 10 μL of the inoculum on the test samples of VITRO-SKIN®˜[10⁶ CFU].
8. Spread bacteria out over the center of the VITRO-SKIN® using the side and tip of the pipette tip.
9. Allow bacteria to dry onto the VITRO-SKIN® for 2 hours at 37° C. in a humidified dessicator in the incubator.
10. Place 5 mL BPW into each 3″×5″ sterile stomacher bag (labeled with the sample).
11. Using sterile T-pins, pin the VITRO-SKIN® samples onto the PVC pipe test vehicle at the prepared spaces using pre-drilled holes in the pipe.
12. Take 2 pieces of inoculated VITRO-SKIN® samples and stomach them directly without running them through the cabinet.
13. For each test, hang the test vehicle on the shackle of the conveyor.
14. Run test article through the cabinet at the desired speed.
15. Remove test article from shackle.
16. Carefully remove the pins and, using sterile forceps, place VITRO-SKIN® into stomacher bag containing 5 mL sterile BPW.
17. Close and massage stomacher bag for 30 seconds by hand.
18. Plate either 1.0 mL or 20 μL of stomacher bag contents, with 1:10 dilutions if necessary, on TSA plates.
19. Incubate TSA plates overnight for 18-24 hours at 37° C.
20. Clean all surfaces and test article

### Day 3

21. Enumerate Colonies

3.1.4 Test 1 Results

In a first test, the above test method was replicated seven times for four different configurations including two different treatment water types and two different conveyor rates. In a first “water only” configuration, the pump was activated but the electrolysis cells and the electroporation electrodes were deactivated. As such, each test piece was rinsed in rinse cabinet 500 by regular tap water delivered by the nozzles 520. In a second “treated water” configuration, the electrolysis cells and the electroporation electrodes were also activated. As such, each test piece was treated in rinse cabinet 500 by electrochemically-activated water, which also conducted an alternating electric field.

Also, for each treatment water type, the conveyor was operated at two different rates: a first rate in which the duration of contact time of the test vehicle with the spray output was 4 seconds, and a second rate in which the duration of contact time of the test vehicle with the spray output was 24 seconds.

To conduct the tests, two power supplies were used to drive the control circuit that applied the voltage potential to the electroporation electrodes 554 tap water delivered by the nozzles 520. As a result, the stepped-up voltage at the transformer output (see FIG. 8) was about 2 kV peak-to-peak. Being a simple bench test, the voltage output had a sinusoidal shape without the local peak discontinuities shown in the waveform of FIG. 6.

For each of the seven iterations in the first and second configurations, the control pieces of VITRO-SKIN® indicated each test piece contained an average of 4.61 log₁₀ Colony Forming Units (CFUs) prior to treatment by rinse cabinet 500.

The test results are shown below:

| Inoculated | | | Average Bacterial Count | | | |
|---|---|---|---|---|---|---|
| Control(log₁₀) | | | Following Treatment (over | | | |
| prior to | Itera- | Treatment | 7 iterations) ± 1 standard | | | |
| treatment | tions | Liquid | deviation | | | |
| Spray Time | | | 4 seconds | | 24 seconds | |
| 4.61 CFUs | 7 | Water Only | 3.1 | ± 0.23 | 1.97 | ± 0.4 |
| 4.61 CFUs | 7 | Activated Water | 3.18 | ± 0.20 | 2.74 | ± 0.37 |
| | | with E-Field | | | | |

| Log₁₀ Reduction Water only vs. Activated | 0.08 | 0.77 |
|---|---|---|
| Water | | |

The above results show that, for a 4 second treatment time, the activated spray output achieved a 0.08 log₁₀ reduction in bacterial count as compared to plain, untreated water.

For a 24 second treatment time, the activated spray output achieved a 0.77 log₁₀ reduction in bacterial count as compared to plain, untreated water.

3.1.5 Test 2 Results

A second test was performed on the same day as the first test (Test 1), which was identical as the first test except that four power supplies were connected together in parallel for supplying power to the transformer, which resulted in an output voltage from the transformer of about 4 kV to the electroporation electrodes.

The test results are shown below:

| Inoculated | | | Average Bacterial Count | | | |
|---|---|---|---|---|---|---|
| Control (log₁₀) | | | Following Treatment (over | | | |
| prior to | Itera- | Treatment | 7 iterations) ± 1 standard | | | |
| treatment | tions | Liquid | deviation | | | |
| Spray Time | | | 4 seconds | | 24 seconds | |
| 4.46 CFUs | 7 | Water Only | 3.41 | ± 0.32 | 2.06 | ± 0.25 |
| 4.46 CFUs | 7 | Activated Water | 3.47 | ± 0.66 | 3.58 | ± 0.34 |
| | | with E-Field | | | | |

| Log₁₀ Reduction Water only vs. Activated | 0.06 | 1.52 |
|---|---|---|
| Water | | |

The above results show that, for a 4 second treatment time, the activated spray output achieved a 0.06 log₁₀ reduction in bacterial count as compared to plain, untreated water.

For a 24 second treatment time, the activated spray output achieved a 1.52 log₁₀ reduction in bacterial count as compared to plain, untreated water.

3.1.6 Test 3 Results

In a third test, the above test method was replicated fifteen times for two different treatment water types and one conveyor rate. In a first “water only” configuration, the pump was activated but the electrolysis cells and the electroporation electrodes were deactivated. As such, each test piece was rinsed in rinse cabinet 500 by regular tap water delivered by the nozzles 520. In a second “treated water” configuration, the electrolysis cells and the electroporation electrodes were also activated. As such, each test piece was treated in rinse cabinet 500 by electrochemically-activated water, which also conducted an alternating electric field.

Also, for each treatment water type, the conveyor was operated at a rate in which the duration of contact time of the test vehicle with the spray output was 12 seconds.

To conduct the tests, four power supplies were used in parallel to drive the control circuit that applied the voltage potential to the electroporation electrodes 554 tap water delivered by the nozzles 520. As a result, the stepped-up voltage at the transformer output (see FIG. 8) was about 4 kV peak-to-peak. Being a simple bench test, the voltage output had a sinusoidal shape without the local peak discontinuities shown in the waveform of FIG. 6.

For each of the seven iterations in the first and second configurations, the control pieces of VITRO-SKIN® indicated each test piece contained an average of 6.06 log₁₀ Colony Forming Units (CFUs) prior to treatment by rinse cabinet 500.

The test results are shown below:

| Inoculated | | | Average Bacterial Count | |
|---|---|---|---|---|
| Control (log₁₀) | | | Following Treatment (over | |
| prior to | Itera- | Treatment | 7 iterations) ± 1 standard | |
| treatment | tions | Liquid | deviation | |
| Spray Time | | | 12 seconds | |
| 6.06 CFUs | 15 | Water Only | 2.2 | ± 0.27 |
| 6.06 CFUs | 15 | Activated Water | 2.76 | ± 0.48 |
| | | with E-Field | | |

| Log₁₀ Reduction Water only vs. Activated | 0.55 |
|---|---|
| Water | |

The above results show that, for a 12 second treatment time, the activated spray output achieved a 0.55 log₁₀ reduction in bacterial count as compared to plain, untreated water.

3.1.7 Test 4 Results

In a fourth test, the above test method was identical to that described for Test 3.

The test results are shown below:

| Inoculated | | | Average Bacterial Count | |
|---|---|---|---|---|
| Control (log₁₀) | | | Following Treatment (over | |
| prior to | Itera- | Treatment | 7 iterations) ± 1 standard | |
| treatment | tions | Liquid | deviation | |
| Spray Time | | | 12 seconds | |
| 6.09 CFUs | 15 | Water Only | 1.70 | ± 0.35 |
| 6.09 CFUs | 15 | Activated Water | 2.41 | ± 0.85 |
| | | with E-Field | | |

| Log₁₀ Reduction Water only vs. Activated | 0.71 |
|---|---|
| Water | |

The above results show that, for a 12 second treatment time, the activated spray output achieved a 0.71 log₁₀ reduction in bacterial count as compared to plain, untreated water.

Because the “water only” and the “activated water” were sprayed onto their respective test strips and the “activated water” achieved a significant log₁₀ reduction in bacterial count relative to “water only”, it is believed that the properties of the electrolyzed water and the electric field applied through the spray output attributed to the increased cleaning and sanitizing capabilities of rinse cabinet 500.

Although the present disclosure has been described with reference to one or more embodiments, workers skilled in the art will recognize that changes may be made in form and detail without departing from the scope of the disclosure and/or the issued claims appended hereto. Also while certain embodiments and/or examples have been discussed herein, the scope of the invention is not limited to such embodiments and/or examples. One skilled in the art may implement variations of these embodiments and/or examples that will be covered by one or more issued claims appended hereto.

What is claimed is:

1. An apparatus comprising:
a livestock carcass travel path;
at least one liquid dispenser configured to dispense liquid to the carcass travel path;
at least one treatment electrode; and
a control circuit configured to cause an alternating electric field to be generated between the electrode and a surface of a carcass along the travel path, through the dispensed liquid.

2. The apparatus of claim 1, wherein the control circuit lacks a corresponding return electrode for the treatment electrode, wherein the control circuit is configured such that the carcass serves as a circuit ground for the alternating electric field with respect to the treatment electrode.

3. The apparatus of claim 1, wherein the control circuit is configured to apply an alternating voltage potential to the treatment electrode having a frequency in a range of about 20 kilohertz to about 800 kilohertz and a voltage of about 2 kV to about 20 kV peak-to-peak.

4. The apparatus of claim 3, wherein:
the frequency is in a range selected from the group consisting of between 20 kHz and 100 kHz, between 25 kHz and 50 kHz, between 30 kHz and 60 kHz, between 28 kHz and 40 kHz, and about 30 kHz.

5. The apparatus of claim 1 and further comprising:
a liquid flow path, from a liquid source through the liquid dispenser; and
an output liquid travel path from the liquid dispenser to the carcass travel path,
wherein the treatment electrode is positioned to make electrical contact with feed liquid traveling along at least one of the liquid flow path or the output spray travel path.

6. The apparatus of claim 5, wherein the liquid dispenser comprises a spray nozzle and the treatment electrode is secured to the spray nozzle.

7. The apparatus of claim 1, further comprising:
a liquid flow path, from a liquid source through the liquid dispenser; and
an electrolysis cell in the liquid flow path and comprising electrolysis cell electrodes separated from one another by a gap, wherein the electrolysis cell electrodes are distinct from the treatment electrode.

8. The apparatus of claim 7, wherein the electrolysis cell comprises a barrier positioned in the gap between the electrolysis cell electrodes, wherein the barrier has pores having diameters selected from the group consisting of a range of 100 microns to 200 microns, and a range of 100 microns to 110 microns.

9. The apparatus of claim 8, wherein the electrolysis cell produces an anolyte and a catholyte and wherein the treatment electrode is positioned to apply the alternating potential to at least one of the following, which is dispensed from the liquid dispenser:
the anolyte;
the catholyte; or
a combination of the anolyte and the catholyte.

10. The apparatus of claim 7, wherein the apparatus comprises a further control circuit which is configured to apply a DC voltage to the electrolysis cell electrodes in a range of 5 volts to 60 volts.

11. The apparatus of claim 1, wherein the apparatus further comprises:
a conveyor extending along the travel path; and
a poultry shackle carried by the conveyor and configured to carry a poultry carcass.

12. The apparatus of claim 11, wherein the apparatus further comprises:
a rinse cabinet comprising the at least one liquid dispenser and the at least one treatment electrode; and
a plucker positioned along the carcass travel path, wherein the conveyor is configured to move poultry carcasses through the plucker and then through the rinse cabinet at a rate such that each carcass is contacted by liquid dispensed from the liquid dispenser within a time period of greater than zero seconds and less than or equal to 30 seconds.

13. The apparatus of claim 11, wherein:
the at least one liquid dispenser comprises a plurality of liquid dispensers arranged along the carcass travel path, each liquid dispenser having a corresponding treatment electrode and being positioned such that the plurality of liquid dispensers maintain consistent contact between a carcass and a liquid output of at least one of the liquid dispensers as the carcass moves along a section of the carcass travel path within the rinse cabinet; and
the conveyor is configured to move the carcass along the carcass travel path at a rate such that the plurality of liquid dispensers maintain the consistent contact between the carcass and the liquid output for a time period of 6 seconds to 24 seconds.

14. The apparatus of claim 1, wherein the apparatus further comprises one or more liquid travel paths and wherein:
the at least one liquid dispenser comprises first and second sets of spray nozzles on first and second opposing sides of the carcass travel path, each spray nozzle in the first and second sets being coupled to at least one of the liquid travel paths and being oriented to direct a respective spray output toward the carcass travel path; and
the at least one treatment electrode comprises a respective treatment electrode for each of the spray nozzles in the first and second sets, wherein each treatment electrode is electrically coupled to at least one of the respective liquid travel path or the respective spray output; and
the control circuit is electrically coupled the treatment electrodes to generate the alternating electric field between each of the treatment electrodes and the surface of the carcass, through the spray outputs.

15. The apparatus of claim 1, wherein the apparatus further comprises:
a plurality of liquid dispensers positioned on opposing sides of the carcass travel path, each liquid dispenser comprising a spray nozzle oriented toward the carcass travel path and positioned within a range of three inches to six inches of the carcass travel path.

16. A method comprising:
receiving a livestock carcass along a travel path;
dispensing a liquid from at least one liquid dispenser to the carcass along the travel path, so as to create an electrically conductive path from the liquid dispenser to the carcass; and
during the step of dispensing, generating an alternating electric field through the liquid along the conductive path, wherein the electric field is applied to the liquid with a treatment electrode and is sufficient to destroy at least one microorganism on a surface of the carcass.

17. The method of claim 16, wherein the alternating electric field is generated with a control circuit that is coupled to the treatment electrode and lacks a corresponding return electrode for the treatment electrode, wherein the control circuit is configured such that the carcass serves as a circuit ground for the alternating electric field with respect to the treatment electrode.

18. The method of claim 16, wherein the alternating electric field is generated by applying an alternating voltage potential to the treatment electrode having a frequency in a range of about 20 kilohertz to about 800 kilohertz and a voltage of about 2 kV to about 20 kV peak-to-peak.

19. The method of claim 18, wherein:
the frequency is in a range selected from the group consisting of between 20 kHz and 100 kHz, between 25 kHz and 50 kHz, between 30 kHz and 60 kHz, between 28 kHz and 40 kHz, and about 30 kHz.

20. The method of claim 16, wherein:
dispensing comprises receiving the liquid from a liquid flow path; and
generating comprises positioning the treatment electrode to make electrical contact with the liquid along at least one of the liquid flow path or the electrically conductive path created by the liquid between the liquid dispenser and the carcass.

21. The method of claim 20, wherein the liquid dispenser comprises a spray nozzle and the alternating electric field is generated by applying an alternating voltage potential to the spray nozzle.

22. The method of claim 16, and further comprising:
electrolyzing a source liquid with an electrolysis cell prior to the step of dispensing to produce an electrochemically activated liquid, wherein the electrolysis cell comprises electrolysis cell electrodes separated from one another by a gap, which are distinct from the treatment electrode; and
wherein the step of dispensing comprises dispensing the electrochemically activated liquid, through which the alternating electric field is created.

23. The method of claim 22, wherein the electrolysis cell comprises a barrier positioned in the gap between the electrolysis cell electrodes.

24. The method of claim 22, wherein the electrolysis cell produces an anolyte and a catholyte and wherein the treatment electrode is positioned to apply the alternating potential to at least one of the following, which is dispensed from the liquid dispenser:
the anolyte;
the catholyte; or
a combination of the anolyte and the catholyte.

25. The method of claim 22, wherein electrolyzing comprises applying a DC voltage to the electrolysis cell electrodes in a range of 5 volts to 60 volts.

26. The method of claim 16, wherein:
the livestock carcass comprises a poultry carcass;
receiving comprises receiving the poultry carcass from a plucker along a conveyor, which includes a shackle from which the poultry carcass hangs along the carcass travel path; and
within a time period of greater than zero seconds and less than or equal to 30 seconds after the poultry carcass leaves the plucker, contacting the poultry carcass with the liquid dispensed from the at least one liquid dispenser and with the alternating electric field conducted through the dispensed liquid.

27. The method of claim 16, wherein the method further comprises:
maintaining consistent contact between the carcass and the liquid dispensed from the at least one liquid dispenser as the carcass moves along a section of the travel path; and
moving the carcass along the travel path at a constant rate, wherein the rate is selected such that the at least one liquid dispenser maintains the consistent contact between the carcass and the dispensed liquid for a time period of 6 seconds to 24 seconds.

28. The method of claim 16, wherein the method further comprises:
positioning each of the liquid dispensers within a range of three inches to six inches of the carcass travel path.

29. The method of claim 16, wherein the method further comprises:
suspending the at least one microorganism from the surface of the carcass by at least one of the group consisting of charged nanobubbles or a detergent, delivered to the surface by the liquid.

30. The method of claim 16, wherein the electric field is sufficient to cause irreversible electroporation of the microorganism.

31. A poultry rinse cabinet comprising:
a poultry carcass travel path extending through the rinse cabinet;
at least one liquid flow path;
first and second sets of spray nozzles on first and second opposing sides of the carcass travel path, each spray nozzle in the first and second sets being coupled to at least one of the liquid flow paths and being oriented to direct a respective spray output toward the carcass travel path;
a respective treatment electrode for each of the spray nozzles in the first and second sets, wherein each treatment electrode is electrically coupled to at least one of the respective liquid travel path or the respective spray output; and
a control circuit configured to cause an alternating electric field to be generated between each of the treatment electrodes and the carcass travel path, through the respective spray outputs, which is sufficient to destroy at least one microorganism on a surface of a carcass along the travel path.
